(19) United States
(12) Patent Application Publication
(10) Pub. No.: US 2009/0317419 A1
(43) Pub. Date: Dec. 24, 2009

(54) Tetramers

(75) Inventors: Barton F. Haynes, Durham, NC (US); M. Anthony Moody, Durham, NC (US); Laurent Verkoczy, Durham, NC (US); S. Munir Alam, Durham, NC (US); Hua-Xin Liao, Durham, NC (US)

Correspondence Address: NIXON & VANDERHYE, PC, 901 NORTH GLEBE ROAD, 11TH FLOOR, ARLINGTON, VA, 22203, US

(73) Assignee: Duke University, Durham, NC (US)

(21) Appl. No.: 12/320,709

(22) Filed: Feb. 2, 2009

Related U.S. Application Data
(60) Provisional application No. 61/006,808, filed on Jan. 31, 2008.

(51) Int. Cl. A61K 39/21; C07K 7/08; C12N 5/00; C12Q 1/70

(52) U.S. Cl. 424/188.1; 530/326; 435/325; 435/5

(57) ABSTRACT

The present invention relates, in general to human immunodeficiency virus (HIV), and, in particular, to B cell tetramers and to methods of using same for diagnosis, disease monitoring and vaccine development.

This application claims priority from U.S. Provisional Application No. 61/006,808, filed Jan. 31, 2008, the entire content of which is incorporated herein by reference.

This invention was made with government support under Grant No. AI0678501 awarded by the National Institutes of Health. The government has certain rights in the invention.

### TECHNICAL FIELD

The present invention relates, in general to human immunodeficiency virus (HIV), and, in particular, to B cell tetramers and to methods of using same for diagnosis, disease monitoring and vaccine development.

### BACKGROUND

Most highly effective vaccines have induction of neutralizing antibodies as their immune correlates of protection (Plotkin and Plotkin, Pediatric Infectious Disease Journal 20:63-75 (2001)). Successful vaccines currently in use have been developed by attenuating pathogens (e.g., polio, measles, rubella, yellow fever, varicella-zoster), by pathogen or toxin inactivation (e.g., polio, Clostridium tetani, Corynebacterium diphtheriae), by using pathogen subunits (e.g., hepatitis B virus surface antigen, Streptococcus pneumoniae polysaccharide), or by conjugation of a portion of a pathogen to an immunogenic carrier (e.g., Haemophilus influenzae type B, S pneumoniae conjugates). It was not necessary to study specific pathogen molecular epitopes or to understand the origin and regulation of B cells capable of producing protective anti-pathogen antibodies to make these vaccines. Rather, immunization with vaccine candidates was highly effective in inducing long-lasting levels of anti-pathogen neutralizing antibodies (Amanna et al, N. Engl. J. Med. 357:1903-1915 (2007)).

Vaccinologists are currently working to develop the enabling technology to make a preventive HIV-1 vaccine. Unfortunately, efforts to make an effective vaccine using killed HIV-1 (LaCasse et al, Science 283:357-362 (1999), Nunberg, Science 283(5400):357-362 (1999), Science 296:1025 (2002)), attenuated HIV-1 Baba et al, Nat. Med. 5:194-203 (1999)), HIV-1 toxin (Tat) inactivation (Silvera et al, J. Virol. 76:3800-3809 (2002), Pauza et al, PNAS 97:3515-3519 (2000)) and HIV-1 subunit vaccines (VanCott et al, Journal of Immunology 155:4100-4110 (1995), Pitisuttihum et al, Journal of Acquired Immune Deficiency Syndromes 37:1160-1165 (2004)) have to date been unsuccessful. HIV-1 infection is particularly difficult to prevent because of the remarkable ability of HIV-1 to mutate leading to extreme viral diversity (Korber et al, British Medical Bulletin 58:19-42 (2001), Richman et al, Proc. Natl. Acad. Sci. USA 100:4144-4149 (2003)), the immunosuppressive properties of the virus (Masur et al, Annals of Internal Medicine 111:223 (1989)), and the ability of HIV-1 to integrate into the host genome Schroder et al, Cell 110:521-529 (2002)).

Rare broadly neutralizing anti-envelope human monoclonal antibodies (mAbs) have been isolated from HIV-1 infected patients that, when passively administered to non-human primates, have prevented infection with simian-human immunodeficiency virus (SHIV) challenge (Mascola et al, Nature Medicine 6:207-210 (2000), Baba et al, Nature Medicine 6:200-206 (2000)). Unfortunately, no HIV-1 envelope (Env) vaccine candidates have induced these types of antibodies (Burton et al, Nat. Immunol. 5:233-236 (2004)). Rather, HIV-1 Env immunogens preferentially induce antibodies that either are non-neutralizing or neutralize only a limited spectrum of Tier 1 (the more easy to neutralize) HIV-1 strains (Li et al, J. Virol. 80:1414-1426 (2006), Zhang et al, Proc. Natl. Acad. Sci. USA 104:10193-10198 (2007)).

HIV-1 Env does have areas of conserved vulnerability to which antibodies could bind and broadly neutralize HIV-1 if such antibodies could be induced. These sites include carbohydrates on the Env surface (Sanders et al, Journal of Virology 76:7293-7305 (2002)), the region of the gp120 CD4 binding site defined by the broadly neutralizing antibody IgG1b12 (Roben et al, J. of Virology 68:4821-4828 (1994), and membrane proximal Env regions of gp41 defined by the rare human mAbs 2F5, 4E10 and Z13 (Muster et al, Journal of Virology 67:6642-6647 (1993), Stiegler et al, AIDS Research & Human Retroviruses 17:1757-1765 (2001), Zwick et al, Journal of Virology 75:10892-10905 (2001)). Thus far, vaccine candidates that express these epitopes have been antigenic, binding to the broadly neutralizing mAbs with nM affinities, but have not been immunogenic in animals or man (Burton et al, Nat. Immunol. 5:233-236 (2004)).

The reasons postulated for poor immunogenicity of the HIV-1 Env are numerous including glycan shielding of Env neutralizing epitopes (HIV-1 envelope is >40% carbohydrate by mass) (Wei et al, Nature 422:307-312 (2003)), entropic barriers to neutralizing antibody binding (Kwong et al, Nature 420:678-682 (2002)), and masking or diversion of antibody responses by non-neutralizing antibodies Wyatt et al, J. Virol. 71:9722-9731 (1997), Alam et al, J. Virol. 82:115-125 (2008), Alam et al, Journal of Immunology 178:4424-4435 (2007)). In addition, each of the rare broadly neutralizing antibodies are unusual. MAb 2G12 has a peculiar structure, with Fabs that assemble into interlocked variable heavy chain (V_{H}) domain-swapped dimers (Calarese et al, Science 300:2065-2071 (2003)). Mab 2G12 reacts with Env carbohydrates that are synthesized and modified by host glycosyltransferases and are recognized as self carbohydrates Calarese et al, Proc. Natl. Acad. Sci. USA 102:13372-13377 (2005)). MAbs 2F5, 4E10 and Z13 all have long complementarity determining region 3 (CDR3) loops and react with multiple host antigens including host lipids (Zwick et al, Journal of Virology 75:10892-10905 (2001), Ofek et al, Journal of Virology 78:10724-19737 (2004), Cardoso et al, Immunity 22:163-173 (2005)). Similarly, IgG1b12 also has a long hydrophobic CDR3 (Saphire et al, Science 293:1155-1149 (2001)) and reacts with double stranded DNA (dsDNA) Haynes et al, Science 308:1906-1908 (2005)).

These findings have prompted the hypothesis that HIV-1 has evolved such that sites that could give rise to broadly neutralizing antibodies on HIV-1 Env may mimic host antigenic epitopes and induce antibodies that are susceptible to down-modulation by B cell tolerance mechanisms (Haynes et al, Human Antibodies 14:59-67 (2005)). If this hypothesis is correct, then the specific B cells capable of making broadly neutralizing antibodies to 1b12 footprint within the CD4 binding site, to the membrane proximal region (MPER) of gp41 and to Env carbohydrates will be present in immature B cells before negative selection has taken place and will be rare or absent in B cell populations after negative selection has occurred. To test this hypothesis, antigen-specific reagents capable of binding to antigen-specific B cells that are reflective of HIV-1 Env epitopes defined by broadly reactive neutralizing antibodies are required so that neutralizing antibody epitope-specific B cell receptor positive B cells can be recovered for study of antibody genes.

The present invention results, at least in part, from the development and characterization of a novel panel of antigen-specific B cell tetramers that are specific for HIV-1 Env target epitopes of commonly made anti-HIV-1 antibodies, and, as well, tetramers that mirror the gp41 MPER epitope defined by the broadly neutralizing mAb, 2F5. Disclosed herein are methods for production, purification, and quality control of these reagents. In accordance with the invention, HIV-1 B cell tetramers can be used, for example, in identifying and sorting antigen-specific B cells from HIV-1 infected patients.

### SUMMARY OF THE INVENTION

The present invention relates generally to tetramers. More specifically, the invention relates to B cell tetramers and to methods of using same in identifying and sorting antigen-specific B cells from HIV-1 infected patients. The invention further relates methods of using B cell tetramers to induce an immune response.

Objects and advantages of the present invention will be clear from the description that follows.

### BRIEF DESCRIPTION OF THE DRAWINGS

FIG. 1A-1C. Solid lines show stained samples; gray curves show unstained controls. (FIG. 1A) Cross comparison of tetramer binding to antibody coated beads. No binding of tetramer was seen to beads coated with the control mAbs P3X63/Ag8 and A32. Env gp120 V3 loop mAbs 7B9 and F39F bound only to B.con03 V3 tetramer and 62.19 V3 tetramer but not to their sequence scrambled versions nor to any other tetramers. mAb 7B2 against the Env gp41 immunodominant region bound gp41 ID tetramer and showed a slightly higher background with the sequence scrambled tetramer. mAbs 13H11 and 2F5 bound to Env gp41 MPER tetramer but not the sequence scrambled version. None of the mAbs bound the dsDNA mimic tetramer. (FIG. 1B) Demonstration of surface Ig expression on mAb cell lines. Surface immunoglobulin expression was demonstrated by anti-murine-IgG (P3X63/Ag8 and 13H11) or anti-human-IgG (F39F and 7B2) reagents. (FIG. 1C) Cross comparison of tetramer binding to mAb cell lines. No binding of tetramer was seen to control mAb P3X63/Ag8. Env gp120 V3 loop mAb F39F bound only to B.con03 V3 tetramer and 62.19 V3 tetramer. mAb 7B2 against the Env gp41 immunodominant region bound gp41 ID tetramer and showed a slightly higher background with the sequence scrambled tetramer. mAb 13H11 bound to Env gp41 MPER tetramer. None of the mAbs bound the dsDNA mimic tetramer.

FIG. 2. Titration of tetramer on antibody-coated beads and mAb cell lines. Serial dilutions of tetramer reacted with antibody coated beads or cell lines specific for each tetramer show saturable binding. The light gray solid curve shows unstained beads or cells, the dark gray curve shows each saturated with tetramer. The open gray curves show serial dilutions of tetramer and the solid black line shows concentrations above saturation. Tetramers bound to beads or cell lines with increasing signal until saturation was achieved; excess tetramer did not increase signal.

FIG. 3. Cold competition of tetramer on antibody-coated beads and mAb cell lines. The light gray solid curve shows unstained samples, the dark grey solid curve shows APC-labeled tetramer staining in the absence of competing unlabeled tetramer, the solid black line shows APC-tetramer staining with the presence of competing unlabeled tetramer. In each case, complete inhibition of binding was seen.

FIGS. 4A-4D. Detection of small populations of tetramer positive cells. A mixture of anti-gp41 MPER 13H11 cells and anti-gp120 V3 F39F cells was made and then tenfold reductions of 13H11 cells made into using additional F39F cells. FIG. 4A shows unstained cells; FIG. 4B shows the same cell mixture stained with gp41 MPER tetramer. FIGS. 4C and 4D show the serial tenfold reductions stained with gp41 MPER tetramer.

FIG. 5. Lack of cross competition of anti-Ig and tetramer binding. Mixtures of anti-gp120 V3 F39F cells and control P3X63/Ag8 cells were stained singly and in combination with anti-human IgG and 62.19 V3 tetramer. The three upper panels show unstained cells, the anti-human IgG labeled cells, and tetramer labeled cells. The three lower panels show dual staining, performed sequentially in the two left panels and simultaneously in the right panel. In each case no cross competition of the reagents was seen.

FIGS. 6A-6D. (FIG. 6A) Sorting of B cells from a chronically HIV-1 infected patient. In the left panel, PBMC depleted for non-B cells were stained with antibodies without tetramer and used to set the tetramer sorting gate. In the middle panel non-B cell depleted PBMC stained with antibodies and gp41 MPER tetramer were sorted into tetramer positive (red (upper) gate) and tetramer negative (light blue (lower) gate) populations. The right panel shows reanalysis of the sorted cells with enrichment of the tetramer positive cells from 0.15% to 45.3% and complete depletion in the tetramer negative sorted cells. (FIG. 6B) ELISA of secreted antibody from cells cotransfected with recovered Ig genes. Supernatant from culture of cell cotransfected with Ig genes isolated from tetramer positive single sorted cells were reacted with gp41 MPER tetramer or scrambled tetramer bound to the plate. Antibodies showed variable reactivity with gp41 MPER tetramer with three antibodies indicated by arrows having substantial signal over the scrambled tetramer control. Ig genes isolated from a 2F5-secreting cell line as a positive control showed strong reactivity. (FIG. 6C) Binding of alanine substituted gp41 MPER tetramers with 2F5. Twenty variants of gp41 MPER tetramer prepared by single amino acid substitution were studied by binding to antibody coated beads (gray bars) and by SPR (white bars). Most variants showed positive signal in both assays, however, the A13 and A15 variants have no binding to 2F5 mAb in either assay. (FIG. 6D) Binding of alanine substituted gp41 MPER tetramers with 13H11. There is greatly reduced binding of the A14, A18, and A20 variants to 13H11 mAb.

FIG. 7. Binding Ab titers to the 2F5 epitope in BALB/c and C57BL/6 mice over the course of immunizations with 2F5 epitope-containing HR2 peptide T20/DP178Q16. Binding titers from sera obtained from BALB/c and C57BL/6 mice bled prior to immunizations (pre-bleed) or 10 days after each boost (post-immune; P.I.#s 1-5) were measured against plate-bound 2F5 nominal epitope peptide in a standard ELISA. Endpoint titers were determined as the reciprocal of the highest serum dilution assayed against control 2F5 Abs, giving optical density readings of experimental samples ≧0.3.

FIGS. 8A and 8B. Comparison of gp41 2F5 MPER interactions with total splenic B cell populations from naïve BALB/c and C57BL/6 strains. (FIG. 8A) Representative staining of splenocytes from unimmunized C57BL/6 and BALB/c mice (left and right panels, respectively) with APC-labeled gp41 2F5 MPER or 62.19 V3 epitope-specific tetramers (upper and lower panels, respectively). (FIG. 8B) Cold inhibition assay of gp41 2F5 MPER tetramer binding in B220⁺ cells using a 10-fold molar excess of pre-incubation for 30 min. with unlabeled gp41 2F5 MPER or scrambled gp41 2F5 MPER tetramer as competition. Shown are representative histograms showing no competition (left two panels), or competition with a 10-fold molar excess of scrambled gp41 2F5 MPER or gp41 2F5 MPER (middle right and far right panels, respectively). Numbers above gates represent the percentage of gp41 MPER-reactive B cells within the total (B220⁺) B cell population.

FIG. 9. Comparison of B cell interactions with various HIV-1 epitope-bearing B cell tetramers in total B cell splenic populations from naïve BALB/c mice. Graphical representation of surface staining of splenocytes from naïve BALB/c mice using gp120 V3 loop, gp41 immunodominant region, gp41 MPER, and anti-dsDNA epitope-specific B cell tetramers or their respective scrambled counterparts. Plotted are the percentages of tetramer-reactive B cells within the live total splenic B cell population from a representative experiment.

FIGS. 10A-10C. The gp41 2F5 MPER epitope interacts with a significant subset of naïve BALB/c splenic B cells by specifically binding the B Cell Receptor (BCR). (FIG. 10A) Representative surface staining of splenocytes from unimmunized BALB/c mice with gp41 2F5 MPER epitope B cell tetramers, either without any pre-treatment (left panels), with pre-treatment at 37° C. with 10 μg/ml anti-Ig (middle), 5 mg/ml mannan (middle right), or 125 mM α-methyl-mannopyranoside (far right). Numbers below gates represent the percentage of gp41 MPER-reactive B cells within the total (B220⁺) B cell population. (FIG. 10B) Representative kinetics of anti-Ig mediated internalization in purified splenic B cells from naïve BALB/c mice. Results were plotted by calculating the residual % of surface-bound APC-labeled gp41 2F5 MPER epitope scrambled or normal tetramers (gp41 2F5 MPER scr. and gp41 2F5 MPER) remaining on B cells after various periods of 10 μg/ml anti-Ig stimulation at 37° C. or 4° C. (FIG. 10C) Immunofluorescence microscopy of cap formation of anti-Ig/gp41 2F5 MPER B cell tetramer co-localization in purified naïve splenic B cells after BCR cross-linking with an equal mixture of anti-Ig and gp41 MPER epitope B cell tetramers (10 μg/ml). An example of a capped B cell after 15 min of anti-Ig stimulation is shown, either singly as individual immunofluorescent stains of Alex488 Wheat Germ Agglutinin (WGA), APC gp41 2F5 MPER tetramers, R-PE-anti IgM+IgG, or merged.

FIGS. 11A and 11B. Analysis of IgH allotype and V_{H} families involved in gp41 2F5 MPER epitope interactions with the BCR in naïve mice. (FIG. 11A) Representative surface staining of splenocytes from unimmunized C57BL/6 and BALB/c inbred strains (congenic with respect to IgH^{a} and IgH^{b} allotypic determinants) with APC-labeled B cell tetramers bearing either the gp41 2F5 MPER epitope (top panels) or the 62.19 V3 epitope (lower panels). Similar results were seen in B cells from other tissues (not shown). (FIG. 11B) Comparison of V_{H} family usage in gp41 2F5 MPER tetramer (+) and (−) bulk-sorted total splenic B cell repertoires. Total, pre-purified splenic B cell populations from naïve BALB/c mice were sorted into gp41 2F5 MPER tetramer-reactive (gp41 2F5 MPER+) and gp41 2F5 MPER tetramer non-reactive (gp41 2F5 MPER−) fractions and real-time quantitative PCR analysis from extracted DNA was performed with V_{H} family-specific primers. Results are from two independent PCR amplications from a representative sort.

FIGS. 12A and 12B. Mapping of gp41 2F5 MPER residues required for B cell superantigen binding activity using gp41 2F5 MPER epitope alanine scan mutants. (FIG. 12A) List of peptides (annotated numerically according to position of substitution) used to make mutant tetramers with sequential alanine substitutions (annotated in bold) along the wild type gp41 2F5 MPER epitope. (FIG. 12B) Comparison of gp41 2F5 MPER residues required for binding to BCRs as a B cell superantigen (middle panel) with gp41 2F5 MPER residues required for binding to the neutralizing MAb 2F5 (upper panel) and the non-neutralizing MAbs 13H11 and 5A9 (lower panel), with each mutant tetramer (substituted position shown on the x-axis of each panel) used to measure relative binding. For measuring binding of mutant tetramers to 2F5, 13H11 and 5A9 Mabs, unlabeled versions of the tetramers were used in Surface Plasmon Resonance analysis. For measuring binding of mutant tetramers to BCRs as a B cell superantigen, APC-labeled tetramers were used in surface staining analysis of total splenic B cell populations from naïve BALB/c mice, followed by calculating APC-Mean Fluorescence Intensities (MFI), setting the FMO (staining without tetramer) MFI as baseline (blue line (baseline) in middle panel).

FIGS. 13A-13C. Relative frequencies of gp41 MPER-reactive cells in total B cell fractions and in B cell subsets from primary and secondary lymphoid tissues of naïve BALB/c mice. (FIG. 13A) Graphical representation of relative reactivities of BALB/c total BCR⁺B cell fractions in bone marrow, spleen, blood, and peritoneal cavities to the 2F5 epitope. Reactivities were determined in several independent experiments (using ≧5 mice) as the percentage of gp41 2F5 MPER, scrambled gp41 2F5 MPER, 62.19 V3, or scrambled 62.19 V3 tetramer-binding cells within “tetramer⁺ gates”, defined independently in each tissue by setting the baseline to include events greater than those observed in FMO controls (staining without tetramer). Total BCR⁺ B cell populations in all tissues were gated as singlet, live, lineage-, then as CD 19⁺B220⁺ events (for spleen and PBL), CD19⁺ events (for peritoneal lavage), and as CD19⁺IgM⁺IgD⁺ events (for bone marrow). (FIG. 13B) Representative cell surface staining of 2F5 epitope-bearing tetramers in bone marrow, splenic, and peritoneal B cell subsets. For each subset, shown are bivariate histograms showing forward scatter versus either the APC-labeled scrambled gp41 2F5 MPER tetramer (left panels), or the 2F5 epitope-containing tetramer gp41 2F5 MPER (right panels). Total B cell populations in all tissues were gated as singlet, live, lineage-, B220⁺ and/or CD19⁺ cells and B cell subsets within these total B cell fractions were subsequently identified using published subfractionation schemes (Allman et al, J. Immunol. 167(12):6834-6840 (2001), Gorelik et al, J. Immunol. 172(2):762-766 (2004), Hayakawa et al, Proc. Natl. Acad. Sci. USA 84(5):1379-1383 (1987), Ueda et al, J. Immunol. 178(6):3593-3601 (2007)). The red (vertical) line indicates the negative cutoff point for all FMO (no tetramer) controls, the gates (numbered) in blue were used to calculate the percentages of cells binding scambled gp41 2F5 MPER or gp41 2F5 MPER tetramers within each subset. (FIG. 13C) Graphical representation of relative 2F5 epitope reactivities in bone marrow (black), spleen (red) (dotted) and peritoneal cavity (blue) (double dotted) B cell subsets, as calculated over several flow cytometry experiments as described in B).

FIG. 14. Dual tetramer titration on monoclonal antibody cell lines.

FIG. 15. Dual tetramer titration of human PBMC.

FIG. 16. Dual tetramer of human PBMC.

### DETAILED DESCRIPTION OF THE INVENTION

The present invention relates to B cell tetramers specific for epitopes of HIV-1 (e.g., linear epitopes of HIV-1). The invention further relates to methods of identifying populations of B cells (e.g., C19⁺ B cells) in HIV-infected individuals using such tetramers. The invention also relates to MPER immunogens that can be used to generate a focused MPER B cell response and to compositions comprising same.

The instant invention provides tetramers suitable for use in the identification and isolation of HIV-1 epitope-specific B lymphocytes. Described in Example 1 is a panel of reagents (which includes such tetramers) that can be used to sort HIV-1 envelope-specific B cells from HIV-1 infected patients. The panel includes bare and scrambled tetramers that can be used to rigidly control for specificity. (See Table 1.)

Bare tetramers, comprising, for example, free biotin reacted with APC-labeled streptavidin, can be used to control for streptavidin and APC binding B cells while scrambled tetramers, comprising primary sequence scrambled peptides for each of the HIV-1 epitope sequences, can be used control for non-specific charge- or hydrophobicity-based binding. It will be appreciated from a review of this disclosure that binding pairs other than biotin and streptavidin can be used and that other randomly scrambled sequences can be used.

The availability of reagents of the invention (e.g., those described in Table 1) makes it possible to dissect the B cell response to HIV-1. With the instant panel of reagents, differences between commonly made responses, such as the gp41 immunodominant region, and rare responses, such as neutralizing gp41 MPER antibodies, can be probed at the cellular level. The scarcity of such broadly neutralizing antibodies and the rarity of such antibodies in both immunized and infected subjects has been a major hindrance for the field. The reagents of the invention make it possible to determine the basis for the rarity of these antibodies and also they also provide a means for investigating the immune response to infection and immunization.

The sorting of tetramer-binding B cells can be effected using the methods described in Example 1. RNA can then be isolated from the tetramer positive sorted cells and cDNA libraries produced. Immunoglobulin genes can be amplified and isolated using standard techniques. The isolated genes can then be cloned into expression vectors to produce full length IgG heavy and kappa light chains. Isolated immunoglobulin genes (or nucleic acid sequences encoding same) can be administered (e.g., IV) to individuals exposed to HIV-1 to afford protection or to individuals infected with HIV-1 to effect treatment of the infection.

Examples 1 and 2 include a description of a panel of alanine substituted gp41 MPER tetramers (see Table 8 and FIG. 12A). The A13 and A15 mutants demonstrate minimal reactivity with 2F5, consistent with the results from other mapping studies (Blink et al, J. Exp. Med. 201:545-554 (2005)). The A14, A18, and A20 mutants appear to be specific for the broadly neutralizing gp41 MPER mAb 2F5 and to have little binding to the non-neutralizing gp41 MPER mAb 13H11. As such, this panel of reagents can be used to characterize B cells with surface Ig reactive with each kind of tetramer and thus allow for comparison of cells with those differing specificities to determine if there is an immunological basis for the relative rarity of broadly neutralizing 2F5-like antibodies. Furthermore, these reagents can be used to study vaccine candidates and immunization strategies to determine whether they stimulate the desired response, that is, by allowing B cells to be monitored to determine if B cells that produce protective antibodies have been induced to expand by a vaccine (that is, to distinguish between desired (e.g., 2F5-like) and undesired (e.g., 13H11-like) responses). These reagents can be used to identify populations of cells in clinical samples that produce neutralizing antibodies.

The invention further relates to immunogens, and compositions comprising same, that can be used to induce neutralizing antibodies to HIV. Two distinct types of interactions exist between B cell receptors and the MPER 2F5 epitope, one being a superantigen-like interaction. Using the tetramer mutants described in the Examples, it has been demonstrated that binding of 2F5 occurs in the neutralization-sensitive residues D and W within the core 2F5 epitope ELDKWA. B cell superantigen binding activity maps to residues distinct from the 2F5 neutralization-sensitive sites, mapping to a single N-terminal glutamine residue and three consecutive C-terminal residues (L, W and N), both located outside the core 2F5 epitope. Two of the C-terminal residues (L and W) required for B cell superantigen-binding activity are also required for binding to 13H11 and 5A9, others are unique to either MPER-superantigen or MPER-13H11/5A9 interactions. The mutant tetramer reagents can be used to dissociate B cell populations that interact specifically with 2F5 broadly neutralizing epitopes from those that bind to non-neutralizing and superantigen-specific MPER epitopes using the methods provided in the Examples. B cell populations that interact specifically with 2F5 broadly neutralizing epitopes provide a source of antibodies that can be used prophylactically or therapeutically.

It will be appreciated from a review of FIG. 12 that tetramer A18, for example, can be used as an immunogen to induce 2F5-like antibodies but not non-neutralizing MPER antibodies. Thus, the invention includes a composition comprising such a tetramer and a carrier. The tetramer can be administered to a patient in need thereof, for example, with an adjuvant (e.g., oligoCpG oligonucleotides, squalene adjuvants, TRL-7 and TRL-4 agonists, and liposome adjuvants). Optimum dosing regimens can be established by one skilled in the art without undue experimentation.

In addition to the mutants described in the Examples below, the invention also relates to the following double mutants and tetramers thereof:

| | |
|---|---|
| 2F5-specific | |
| Biotin-GGG-SP62-A18: | QQEKNEQELLELDKWASAWN |
| Biotin-GGG-SP62-A18/A19: | QQEKNEQELLELDKWASAAN |
| 5A9/13H11-specific | |
| Biotin-GGG-SP62-A2/A15: | QAEKNEQELLELDKAASLWN |
| Biotin-GGG-SP62-A15/A19: | QQEKNEQELLELDKAASLAN |
| sAg-specific | |
| Biotin-GGG-SP62-A13/A14: | QQEKNEQELLELAAWASLWN |
| Biotin-GGG-SP62-A13/A20: | QQEKNEQELLELAKWASLWA |
| Biotin-GGG-SP62-A15/A18: | QQEKNEQELLELDKAASAWN |

Such tetramers can be used to identify B cell populations producing neutralizing antibodies in the manner described in the Examples.

The invention further relates to the following 2F5-specific mutant SP62 peptide

A18/A19:
QQEKNEQELLELDKWASAANWF.

The invention relates to compositions comprising this peptide and a carrier. This peptide can be administered (e.g., coupled to a carrier) to an individual in an amount sufficient to induce an immune response (e.g., neutralizing antibodies). The peptide can be administered alone or with an adjuvant (including those described above). Optimum dosing regimens can be determined by one skilled in the art without undue experimentation.

Certain aspects of the invention can be described in greater detail in the non-limiting Examples that follows. (See also WO 2007/112079, U.S. application Ser. No. 12/225,541 and US Published Application 20070086946 which are incorporated herein by reference.)

### Example 1

### Experimental Details

Antibodies and cell lines. Anti-HIV-1 membrane proximal gp41 mAb 2F5 (Buchacher et al, AIDS Research & Human Retroviruses 10:359-369 (1994)) was purchased from Polymun Scientific (Vienna, Austria). The anti-HIV-1 mAbs A32 (gp120) (Wyatt et al, Journal of virology 69:5723-5733 (1995)), F39F (gp120 V3 loop), and 7B2 (gp41 immunodominant region) were produced as described (Robinson et al, AIDS Research & Human Retroviruses 6:567-579 (1990)). The mouse myeloma cell line P3X63/Ag8 was obtained from ATCC. Mouse mAb cell lines 7B9 (Yu et al, Clinical & Vaccine Immunology 13L1204-1211 (2006)) and 13H11 (Alam et al, Journal of Immunology 178:4424-4435 (2007)) were prepared as previously described. Mab cell lines for antibody production A32, F39F, 7B2, P3X63/Ag8, 7B9, and 13H11 were grown in serum-free media and mAbs purified using anti-Ig columns. Antibodies for flow cytometry were anti-human-IgG (H+L)-FITC and anti-mouse-IgG (H+L)-FITC from Kirkegaard & Perry Laboratories, Inc. (Gaithersburg, Md.); anti-human-IgG-PE, CD3-PE-Cy5, CD16-PE-Cy5, and CD19-PE from BD Biosciences (Mountain View, Calif.); and CD14-PE-Cy5 from Caltag/Invitrogen (Carlsbad, Calif.). All antibodies were titered and used at optimal concentrations for flow cytometry.

Peptides. Peptides were synthesized (PrimmBiotech, Inc., Cambridge, Mass.) and purified by reverse phase HPLC. All peptides were assessed for purity by HPLC (>95% pure) and confirmed to be of the correct mass by mass spectrometry (Table 1).

TABLE 1
Sequences of peptides used for tetramer production.

| peptide name | | |
|---|---|---|
| location | peptide sequence* | sequence |
| B.con03 V3 | biotin-GGGTRPNNNTRKSIHIGPGRAFYTTGEIIGDIRQAH | gp120 Env₃₀₆₋₃₃₈ |
| B.con03 V3 | biotin-GGGITIDNIGHHNRITFRAANTRPISGQRGEYPGKT | |
| scrambled | | |
| 62.19 V3 | biotin-GGGTRPNNNTRKSIHIGPGRAFYATE | gp120 Env₃₀₆₋₃₂₈ |
| 62.19 V3 | biotin-GGGNPATISRTIGHNFRYTPAREKNG | |
| scrambled | | |
| gp41 ID | biotin-GGGKQLQARVLAVERYLKDQQLLGIWGCSGKLICTTAV | gp41 Env₅₈₁₋₆₁₅ |
| gp41 ID | biotin-GGGQLDSIQKEVYQLGRQLVWCTARLATKLVGKACGIL | |
| scrambled | | |
| gp41 MPER | biotin-GGGQQEKNEQELLELDKWASLWN | gp41 Env₆₅₉₋₆₇₈** |
| gp41 MPER | biotin-GGGNKEQDQAEESLQLWEKLNWL | |
| scrambled | | |
| dsDNA mimic | biotin-GGGGGDWEYSVWLSN | |

*Env sequences are numbered relative to HIV-1_{HXB2CG} using the system described by Korber, et al. (Human Retroviruses and AIDS (eds. B. Korber et al) III-102-111 (Theoretical Biology and Biophysics Group, Los Alamos National Laboratory, Los Alamos, NM (1998). The B.con03 V3, gp41 ID, and gp41 MPER sequences are identical to the August 2004 clade B consensus sequence available from the Los Alamos National Laboratory HIV Sequence Database (accessible from http://www.hiv.lanl.gov/content/). The 62.19 V3 sequence was obtained from Haynes, et al (Virology 345:44-55 (2006)). The dsDNA mimic peptide sequence was obtained from Gaynor, et al (Proc. Natl. Acad. Sci. USA 94:1955-1 960 (1997)).
**gp41 Env₆₅₉₋₆₇₈ is SP62.

B cell tetramers. Biotinylated peptides were brought to 200 μM in phosphate-buffered saline (PBS)+0.02% NaN₃, mixed 1:1 with APC-conjugated streptavidin (Molecular Probes, Eugene, Oreg.) at 6.1 μM, and incubated (4° C. overnight). Cold tetramer preparations were made in the same manner except that the streptavidin was at 24 μM. Bare tetramer was prepared in the same manner using 200 μM biotin (Fisher Scientific, Fair Lawn, N.J.). Small preparations (<500 μL) were purified by equilibrating Micro Bio-Spin 30 columns (BioRad Laboratories, Hercules, Calif.) with PBS+0.02% NaN₃ before removing excess peptide by gel filtration. Large preparations (>500 μL) were purified by concentration in a Centriprep 30,000Da MWCO concentrator (Millipore, Billerica, Mass.) followed by four 5 mL washes with PBS+0.02% NaN₃. Final tetramer concentrations were determined using UV-Vis absorbance measurements on a NanoDrop spectrophotometer (NanoDrop Technologies, Wilmington, Del.) and calculated as described below or by bulk protein determination using a Micro BCA Protein Assay Kit (Pierce, Rockford Ill.). Each method yielded similar results.

Tetramer Concentration Calculations. Determination of tetramer concentrations can be performed using various techniques including standard colorimetric protein assays (e.g., microplate BCA assay) and by using the absorbance of proteins and fluorochromes. Both of these have been tested and they have been found to be consistent with each other. Colorimetric protein assays are carried out using standard protocols supplied by the manufacturer and will not be discussed further. What follows is the theoretical basis for determination of tetramer concentration using UV-Vis absorbance and sample calculations based on this method.

Absorbance of compounds is described by the Beer-Lambert law (Atkins, P. P. W. in Quanta: A Handbook of Concepts, Edn. 2^{nd} 1 (Oxford University Press, Oxford; 1991) that states

I=I₀10^{-ε[T]l} (Eq. 1)

where I₀ is the incident intensity of light, I is the transmitted intensity, ε is the molar absorption coefficient of the compound in question, [T] is the concentration of the compound in question, and l is the path length of the light through the absorbing medium. Note that the value of ε varies with wavelength and is usually reported for the maximal absorbance wavelength of a given compound. Modern spectrophotometers often report absorbance instead of intensity measurements, thus as a practical matter this equation is often rearranged to use absorbance A defined as

$$A = ɛ [ T ] l = log \frac{{I}_{0}}{I} \qquad ( Eq . 2 )$$

Thus, with the knowledge of the molar absorption coefficient, the path length, and the absorbance value, the concentration of the material of interest can be determined. For streptavidin conjugates an approximate concentration can be obtained by measuring the absorbance at 280 nm and using 171,800M⁻¹ cm⁻¹ as the value for ε₂₈₀. This method assumes a minor contribution of the bound peptides to the absorbance, thus for large peptides and for those with unusual amino acid composition this assumption is not valid.

For proteins with fluorochromes attached this approximation is in error due to both the spectral overlap of the fluorochrome at 280 nm and the lack of a correction for the number of fluorochromes attached to a given protein. This latter value is often referred to as the fluorochrome to protein (F:P) ratio that is designated f. To determine the concentration of a labeled tetramer the value of f must first be determined for a given lot of fluorochrome-labeled streptavidin. This is greatly simplified by the fact that most proteins, including streptavidin, have little absorbance at the absorbance maxima of common fluorochromes. Taking the manufacturer's value for the concentration of the fluorochrome-labeled streptavidin (in mol/L) as [S], the absorbance of this material is measured at the absorbance maximum for the fluorochrome in question (e.g., 652 nm for allophycocyanin, ε₆₅₂=733,200 M⁻¹ cm⁻¹). Using [F] for the concentration of fluorochrome, use can then be made of the following equation to experimentally determine the value of f.

[F]=f[S] (Eq. 3)

The absorbance of the labeled streptavidin is approximately equal to the total absorbance of the fluorochrome label. Combining this with Eq. 2 results in

A_{labeled streptavidin}≈A_{fluorochrome}=ε_{fluorochrome}[F]l (Eq. 4)

and that combined with Eq. 3 gives

A_{labeled streptavidin}=ε_{fluorochrome}f[S]l (Eq. 5)

The concentration of fluorochrome-labeled streptavidin is often supplied in mg/mL rather than in molarity, so this value is assigned the variable C and the molecular weight of the compound is assigned the variable W, resulting in the following equations.

$$\begin{matrix}[ S ] = \frac{{C}_{labeled streptavidin}}{{W}_{labeled streptavidin}} & ( Eq . 6 ) \\ {W}_{labeled streptavidin} = {W}_{streptavidin} + f {W}_{fluorochrome} & ( Eq . 7 )\end{matrix}$$

Combining Eq. 5 and 6 gives

$${A}_{labeled streptavidin} = {ɛ}_{fluorochrome} f \frac{{C}_{labeled streptavidin}}{{W}_{labeled streptavidin}} l \qquad ( Eq . 8 )$$

and combining this with Eq. 7 provides

$${A}_{labeled streptavidin} = \frac{{ɛ}_{fluorochrome} f {C}_{labeled streptavidin} l}{{W}_{streptavidin} + f {W}_{fluorochrome}} . \qquad ( Eq . 9 )$$

Solving this equation for f, the following results

$$f = \frac{{A}_{labeled streptavidin} {W}_{streptavidin}}{\begin{matrix}{ɛ}_{fluorochrome} {C}_{labeled streptavidin} l - \\ {A}_{labeled streptavidin} {W}_{fluorochrome}\end{matrix}} . \qquad ( Eq . 10 )$$

As an example, allophycocyanin-labeled streptavidin at 1 mg/mL was found to have an absorbance of 0.470 at 652 nm with a path length of 0.1 cm. Using the value for ε₆₅₂ from above, 104,000 g/mol for W_{allophycocyanin}, and 55,000 g/mol for W_{streptavidin}, f is calculated as follows.

$$\begin{matrix}f = \frac{( 0.470 ) ( 55 , 000 g \text{/} mol )}{\begin{matrix}( 733 , 200 {M}^{- 1} {cm}^{- 1} ) ( 1 g \text{/} L ) ( 0.1 cm ) - \\ ( 0.47 ) ( 104 , 000 g \text{/} mol )\end{matrix}} \\ = \frac{25 , 850 g \text{/} mol}{73 , 320 g \text{/} mol - 48 , 880 g \text{/} mol} \\ = 1.06\end{matrix} \qquad ( Eq . 11 )$$

This value is consistent with that provided by the manufacturer.

Assuming that the reacting peptide does not have significant absorbance at the absorbance maximum of the fluorochrome, the value of f determined for the reagent streptavidin can be used to calculate the concentration of the tetramer product. Taking Eq. 5 in a slightly different form, it is noted

Aₜₑₜᵣₐₘₑᵣ=ε_{fluorochrome}f[T]l (Eq. 12)

where [T] is the molar concentration of tetramer in solution. Solving this for [T] gives

$$[ T ] = \frac{{A}_{tetramer}}{{ɛ}_{fluorochrome} fl} . \qquad ( Eq . 13 )$$

Finally, Eq. 6 can be used to convert this formula to provide an answer in mg/mL,

$$[ T ] = \frac{{A}_{tetramer}}{{ɛ}_{fluorochrome} fl} = \frac{{C}_{tetramer}}{{W}_{tetramer}} . \qquad ( Eq . 14 )$$

Solving for Cₜₑₜᵣₐₘₑᵣ yields

$${C}_{tetramer} = \frac{{A}_{tetramer} {W}_{tetramer}}{{ɛ}_{fluorochrome} fl} . \qquad ( Eq . 15 )$$

As an example of this calculation, streptavidin labeled with AlexaFluor® 700 (ε₇₀₀=192,000M⁻¹ cm⁻¹) was determined to have a value of f=2.67. This streptavidin conjugate was used to make tetramers with B.con03 V3 peptide and the product's molecular weight was 74,868 g/mol. The product solution was determined to have A₇₀₀=0.252 with l=0.1 cm, and so using Eq. 15 gives

$$\begin{matrix}{C}_{tetramer} = \frac{( 0.252 ) ( 74 , 868 g \text{/} mol )}{( 192 , 000 {M}^{- 1} {cm}^{- 1} ) ( 2.67 ) ( 0.1 cm )} \\ = 0.368 g \text{/} L \\ = 368 µg \text{/} mL .\end{matrix} \qquad ( Eq . 16 )$$

It is important to note that the molecular weight of the fluorochrome-labeled tetramer also depends on the value of f, as noted in Eq. 7 above. Since streptavidin has four binding sites for biotin, four biotinylated peptides will be bound and will contribute to the molecular weight of the final product. The molecular weight of the fluorochrome-labeled tetramer will, therefore, be

Wₜₑₜᵣₐₘₑᵣ=W_{labeled streptavidin}+4W_{biotinylated peptide}=W_{streptavidin}+fW_{fluorchrome}+4W_{biotinylated peptide} (Eq. 17)

Surface Plasmon Resonance (SPR) measurements of tetramer binding to peptide epitopes. Tetramer binding assays were carried out on a BIAcore 3000 (BIAcore/GE Healthcare, Piscataway, N.J.) instrument using mAbs against each specific B cell epitope.

Each mAb (about 1000 RU) was first captured on anti-human Fc mAb (Pierce, Rockford, Ill.) immobilized on a CM5 sensor chip. Approximately 3000 RU of anti-human Fc mAb was covalently coupled to each flow cell of the sensor chip as described before (Alam et al, Journal of Immunology 178:4424-4435 (2007)). Each B cell tetramer was injected over its epitope specific mAb or a control surface immobilized with irrelevant mAb. For each tetramer binding curve, non-specific binding signals obtained from the control surface were subtracted. The binding assay was monitored at 25° C. and using PBS, pH 7.4, as the running buffer. The BIAevaluation 3.0 software (BIAcore/GE Healthcare, Piscataway, N.J.) was used for SPR binding data analyses.

Antibody-coated beads. Carboxyl-polystyrene beads (3.0-3.4 μm) (Spherotech, Inc., Lake Forest, Ill.) were resuspended in 0.01 M sodium acetate buffer (pH 5.0) and reacted (20° C.×1 hr) with 1-ethyl-3-(3-dimethylaminopropyl) carbodiimide hydrochloride (EDC) (Sigma-Aldrich, Co., St. Louis, Mo.) at a ratio of 3.1 mg beads to 1 mg EDC. Beads were washed with sodium acetate buffer, resuspended in a minimal volume before adding purified mAb in the same buffer (ratio of 16 mg beads to 1 mg mAb), incubated (20° C.×2 hrs), then washed with sodium acetate buffer. Beads were resuspended at 5% in PBS containing 1% bovine serum albumin and 0.02% sodium azide.

Use of antibody-coated beads and cell lines for tetramer quality control. Tetramer stocks were diluted to twice the desired final concentration in PBS containing 1% BSA. An equal volume of beads or cells at 5×10⁵ up to 2×10⁶ per sample were mixed with tetramer and incubated (4° C.×30 min). Beads or cells were washed with PBS+1% BSA before being fixed in 2% methanol-free formaldehyde (Polysciences, Inc, Warrington, Pa.) in PBS. Samples were stored at 4° C. prior to being analyzed by flow cytometry (BD LSR II, Becton Dickinson, Mountain View, Calif.). Data were analyzed using FlowJo (Tree Star, Ashland, Oreg.). For tetramer titration 40-80 ng tetramer per 10⁶ cells/beads was serially diluted over 8-12 points. Optimal concentrations determined by this method were used for subsequent experiments. For cold competition experiments the incubation step was performed by staining with 10-fold excess unlabeled tetramer (4° C.×30 min) prior to the addition of APC-labeled tetramer. For cell dilution experiments, a mixture of mAb cell lines was made and then 1:10 serial dilutions of cells prepared. For cross competition experiments, mixtures of cells were prepared and then stained singly with anti-IgG reagents or tetramer, or the cells were doubly stained with both reagents either sequentially or concurrently.

Staining of bulk populations for the sorts. Cells from uninfected or HIV-1 infected patients were obtained by leukopheresis under an IRB-approved protocol and were rested in culture overnight in 10% FBS in RPMI-1640 at 37° C. in 5% CO₂. Rested cells were enriched for B cells by depletion of non-B cells using magnetic bead separation (BD Biosciences, Mountain View, Calif.). Cells were stained with CD3-PE-Cy5, CD14-PE-Cy5, and CD16-PE-Cy5 to label non-B cells, with CD19-PE as a B cell marker, and with bare tetramer, single color tetramer, or dual color tetramers for gp41 MPER tetramer. Cells were sorted on a BD FACS Aria (BD Biosciences, Mountain View, Calif.) into CD3⁻ CD14⁻ CD16⁻ CD19⁺ tetramer and CD3⁻ CD14⁻ CD16⁻ CD19⁺ tetramer⁺ pools and also as single cells into a 96-well plates containing RNA extraction buffer.

Isolation of immunoglobulin genes from sorted tetramer positive single cells by RT and PCR. Tetramer positive single B cells were sorted into wells of 96-well plates, which contained 20 μL/well lysis-reverse transcription buffer consisting of 0.5 μL RNAseOut (Invitrogen, Carlsbad, Calif.), 5 μL 5× first strand cDNA Reaction Buffer, 1.25 μL 10 mM dithiothreitol, 0.065 μL Igepal and 13.25 μL RNase-free H₂O. The reverse transcription (RT) and polymerase chain reaction (PCR) for amplification of the variable regions of heavy (V_{H}), kappa (κ) and lambda (λ) chains of immunoglobulin genes was based on the method described by Tiller et al (Journal of Immunological Methods 329:112-124 (2008)) with the following modifications. Briefly, first strand cDNA was synthesized by incubation (37° C.×1 hr) with Superscript III using primers derived from the constant regions of the heavy chains of human IgG, IgM, IgD and IgA as well as the constant region of human Igκ and Igλ chains (see Table 2). After RT incubation, V_{H}, V_{κ} and V_{λ} genes were amplified separately by first-round PCR using 5 μL of RT products as templates and followed by nested PCR. All PCR was carried in 96-well plates in a 50 L reaction mixture. First PCR contained 5 units of HotStar Taq Plus (Invitrogen, Carlsbad, Calif.), 0.2 mM dNTPs, 0.25 μmol Ig gene specific primers for amplifying all major V_{H} families of heavy chain of IgM, IgG, IgD, IgA1 and IgA2, as well as Igiκ and Igλ (see Table 3). The first round PCR was performed at 95° C.×5 min; 35 cycles of 95° C.×30 sec, 55° C. (V_{H} and V_{κ}) or 50° C. (V_{λ})×60 sec, 72° C.×90 sec; and one cycle of additional extension at 72° C.×7 min. Nested PCR was carried with 2.5 μL of unpurified first round PCR product in a 50 μL reaction mixture containing 5 units of HotStar Taq Plus, 0.2 mM dNTPs, 0.25 μmol Ig gene specific primers for amplifying all major V_{H} families of heavy chain of IgM, IgG, IgD, IgA1 and IgA2, as well as Igκ and Igλ (see Table 4) using a PCR cycling condition of 95° C.×5 min; 35 cycles of 95° C.×30 sec, 58° C. (V_{H}), 60° C. (V_{κ}) or 64° C. (V_{λ})×60 sec, 72° C.×90 sec; and one cycle of additional extension at 72° C.×7 min. Samples of V_{H}, V_{κ} and V_{λ} chain PCR products were analyzed on 1.2% agarose gels. PCR products with the expected size were purified by PCR purification kit (Qiagen, Valencia, Calif.) for sequencing and subquent PCR for transfection. Sequences were analyzed by the IgBlast program (http://www.ncbi.nlm.nih.gov/igblast/) to identify V gene segments and determine the IgH class.

TABLE 2

Primers used for reverse transcriptase
reaction.

| | |
|---|---|
| RT primer | 5′-3′ sequence |

| | |
|---|---|
| IgM-RT | ATG GAG TCG GGA AGG AAG TC |
| IgD-RT | TCA CGG ACG TTG GGT GGT A |
| IgE-RT | TCA CGG AGG TGG CAT TGG A |
| IgA1-RT | CAG GCG ATG ACC ACG TTC C |
| IgA2-RT | CAT GCG ACG ACC ACG TTC C |
| IgG-RT | AGG TGT GCA CGC CGC TGG TC |
| Cκ-newRT | GCA GGC ACA CAA CAG AGG CA |
| Cλ-new-ext | AGG CCA CTG TCA CAG CT |

TABLE 3
Primer pairs used for first round PCR.

| | | | |
|---|---|---|---|
| forward primer | 5′-3′ sequence | reverse primer | 5′-3′ sequence |

| | | | |
|---|---|---|---|
| V_{H}1-Ext | CCA TGG ACT GGA CCT GGA GG | IgA-ext | CGA YGA CCA CGT TCC CAT CT |
| V_{H}2-Ext | ATG GAC ATA CTT TGT TCC A | IgD-ext | CTG TTA TCC TTT GGG TGT CTG CAC |
| V_{H}3-Ext | CCA TGG AGT TTG GGC TGA GC | IgG-ext | CGC CTG AGT TCC ACG ACA CC |
| V_{H}4-Ext | ATG AAA CAC CTG TGG TTC TT | IgM-ext | CCG ACG GGG AAT TCT CAC AG |
| V_{H}5-Ext | ATG GGG TCA ACC GCC ATC CT | | |
| V_{H}6-Ext | ATG TCT GTC TCC TTC CTC AT | | |
| V_{κ}1/2-Ext | GCT CAG CTC CTG GGG CT | Cκ-ext | GAG GCA GTT CCA GAT TTC AA |
| V_{κ}3-Ext | GGA ARC CCC AGC DCA GC | | |
| V_{κ}4/5-Ext | CTS TTS CTY TGG ATC TCT G | | |
| V_{κ}6/7-Ext | CTS CTG CTC TGG GYT CC | | |

TABLE 4
Primer pairs used for first round PCR (continued).

| | | | |
|---|---|---|---|
| Forward primer | 5′-3′ sequence | reverse primer | 5′-3′ sequence |

| | | | |
|---|---|---|---|
| V_{λ}1-Ext | CCT GGG CCC AGT CTG TG | C_{λ}-new-ext | AGG CCA CTG TCA CAG CT |
| V_{λ}2-Ext | CTC CTC ASY CTC CTC ACT | | |
| V_{λ}3-Ext | GGC CTC CTA TGW GCT GAC | | |
| V_{λ}3I-Ext | GTT CTG TGG TTT CTT CTG AGC TG | | |
| V_{λ}4ab-Ext | ACA GGG TCT CTC TCC CAG | | |
| V_{λ}4c-Ext | ACA GGT CTC TGT GCT CTG C | | |
| V_{λ}5/9-Ext | CCC TCT CSC AGS CTG TG | | |
| V_{λ}6-Ext | TCT TGG GCC AAT TTT ATG C | | |
| V_{λ}7/8-Ext | ATT CYC AGR CTG TGG TGA C | | |
| V_{λ}10-Ext | CAG TGG TCC AGG CAG GG | | |

A high throughput strategy was developed for generating Ig gene expression constructs by PCR without cloning based on a method described by Kirchherr et al (Journal of Virological Methods 143:104-111 (2007)) for Env gene expression. A promoter-leader DNA fragment (705 bp) containing a CMV promoter and sequences encoding the Ig leader sequence (METDTLLLWVLLLWVPGSTGD), an IgH-poly A DNA fragment (1188 bp) containing the sequences encoding the constant regions of IgG1 and bovine growth hormone (BGH) poly A tail signal, and an Igκ-poly A or Igλ-poly A DNA fragment (568 bp for Igκ or 536 bp for Igλ) containing the sequences encoding the constant regions of either κ chain or λ chain and BGH poly A tail signal were generated by PCR. PCR was carried out to generate the constructs with all necessary elements including promoter and poly A tail as well as full gene sequences for IgH and IgL chains in a standard 50 μL PCR reaction mixture using 10 ng of promoter-leader DNA fragment, 10 ng of either IgH-poly A DNA fragment, or 10 ng of Igκ-poly A DNA fragment or Igλ-poly A DNA fragment, 10 ng of the purified V^{H}, V_{κ} or V_{λ} genes as templates, and the primer pair of forward primer, HV13220CMV-F262 (AGTAATCAATTACGGGGTCATTAGTTCATAG) and reverse primer, 13248-R1822BGH (TCCCCAGCATGCCTGCTATTGTCTTCCCAATC). The PCR cycling condition was 95° C.×5 min; 25 cycles of 95° C.×30 sec, 55° C.×30 sec, 72° C.×150 sec for IgH or 90 sec for Igκ and Igλ; and one cycle of additional extension at 72° C.×7 min. PCR products were analyzed on 1% agarose gels. PCR products with the expected size (˜2.5 kb for IgH, ˜1.6 kb for Igκ and Igλ) were purified and used for transfection. Human embryonic kidney 293T cells were cultured in 12-well plates and transfected with 2 μg of purified PCR products of Ig heavy and light chain genes derived from the same single cells using PolyFect (Qiagen, Valencia, Calif.) under standard conditions. Cultures were washed 6 hrs after transfection and cultured with 1 mL/well DMEM supplemented with 2% fetal bovine serum for 3 days before supernatants were harvested for analysis by ELISA and Western blot.

ELISA of expressed antibodies. Antibody supernatants were analyzed by ELISA using standard protocols (²⁷. Briefly, gp41 MPER peptide or gp41 MPER scrambled peptide at 0.2 μg/well in 0.1 M NaHCO₃ was coated onto a high-binding ELISA plate (Costar/Corning, Lowell, Mass.) overnight at 4° C. Plates were blocked at RT×2 hrs with PBS containing 4% wt/vol whey protein, 15% goat serum, 0.5% Tween20, and 0.05% NaN₃. 100 μL of supernatant from cultures of cotransfected cells were incubated at RT×2 hrs. Goat-anti-human IgG (heavy- and light-chain)-specific alkaline phosphatase (1:3000 dilution) (catalogue A1543; Sigma, St. Louis, Mo.) diluted in blocking buffer was used as the secondary antibody. The substrate was 2 mM MgCl₂ and 1 mg/mL 4-nitrophenyl phosphate di(2-amino-2-ethyl-1,3-propanediol) salt in 50 mM Na₂CO₃ buffer (pH 9.6) incubated for 18 hrs prior to being read in an ELISA reader at 405 nm.

### Results

Tetramer specificity by surface plasmon resonance studies. A panel of nine B cell tetramer reagents was prepared from biotinylated peptides (Table 1). This panel of tetramers was assayed for specificity by surface plasmon resonance (SPR) for interaction with captured mAbs F39F (anti-gp120 V3), 7B9 (anti-gp120 V3 62.19), 7B2 (anti-gp41 immunodominant region), 2F5 (neutralizing anti-gp41 MPER), 13H11 (non-neutralizing anti-gp41 MPER), 4E10 (anti-distal gp41 MPER), and A32 (anti-conformational gp120). Rate constants for binding (kₒₙ) and dissociation (k_{off}) were calculated and are shown in Table 5. Each tetramer reacted specifically to the relevant mAb; no binding to any mAb tested was seen for any scrambled epitope tetramer nor for the dsDNA mimic tetramer. Further, no binding of any tetramer was seen to the control anti-conformational gp1 20 mAb A32 or to the anti-distal gp41 MPER mAb 4E10. The clade B 62.19 V3 tetramer bound to both the human anti-gp120 V3 mAb F39F and to the murine anti-gp120 V3 mAb 7B9, while the B.con03 V3 tetramer bound only to the human mAb F39F.

TABLE 5
Binding of B cell tetramers to mAbs by surface plasmon resonance.

| | tetramer | | | | |
|---|---|---|---|---|---|
| | B.con03 V3 | 62.19 V3 | gp41 ID* | gp41 MPER | dsDNA mimic |
| mAb | kₒₙ in M⁻¹s⁻¹ × 10³/k_{off} in s⁻¹ × 10⁻³ | | | | |
| F39F (anti-V3) | 83.4/0.65 | 60.5/0.60 | —^{†} | — | — |
| 7B9 (anti-62.19 V3) (Haynes, et al | — | 35.6/0.19 | — | — | — |
| (Virology 345: 44-55 (2006)) | | | | | |
| 7B2 (anti-gp41 immunodominant) | — | — | 1.2/1.1 | — | — |
| 2F5 (anti-MPER neutralizing) | — | — | — | 174/0.37 | — |
| 13H11 (anti-MPER non-neutralizing) | — | — | — | 206/8.1 | — |

*ID = gp41 immunodominant; MPER = gp41 membrane proximal external region; dsDNA = double stranded DNA.
^{†}— = no binding observed. The five mAbs above plus anti-distal gp41mAb 4E10 and anti-conformational gp120 mAb A32 were tested against all nine tetramer constructs and no binding was observed for any combinations other than those shown above. Specifically, no binding of any mAb was seen for the B.con03 V3 Scr, 62.19 V3 Scr, gp41 ID Scr, gp41 MPER Scr, or dsDNA mimic tetramers.

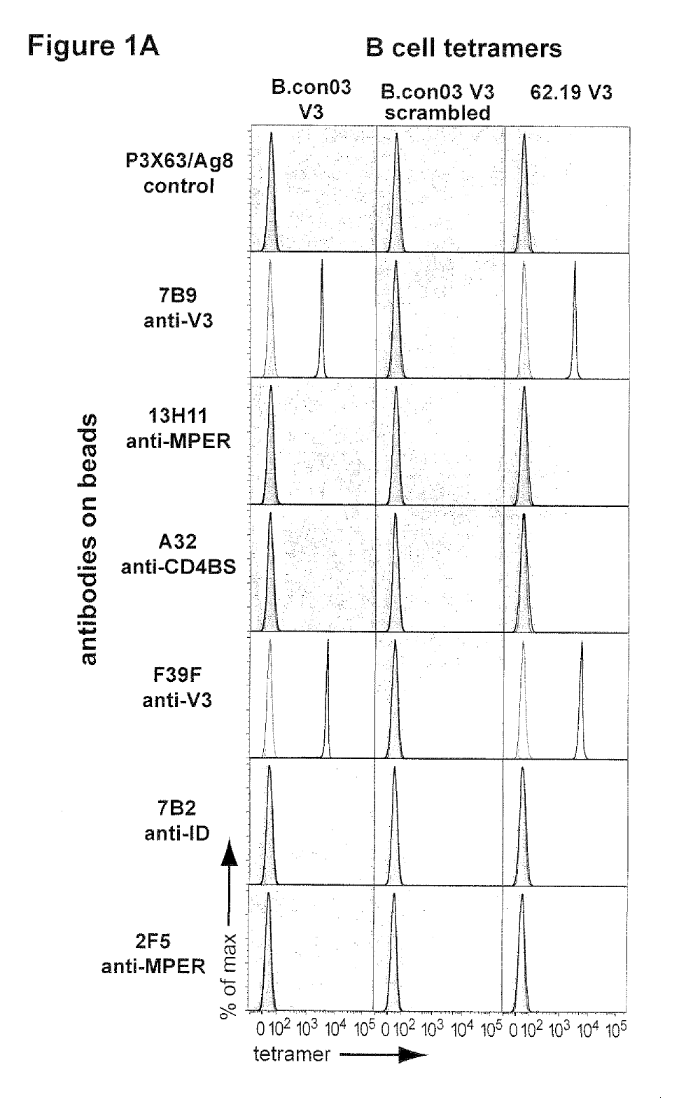

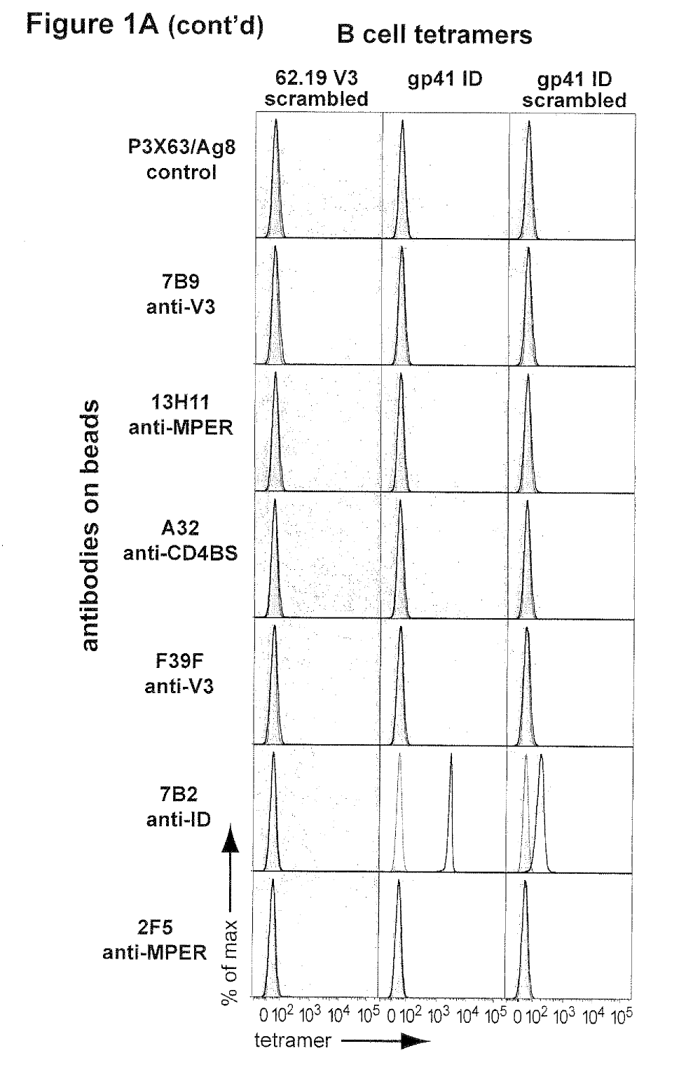

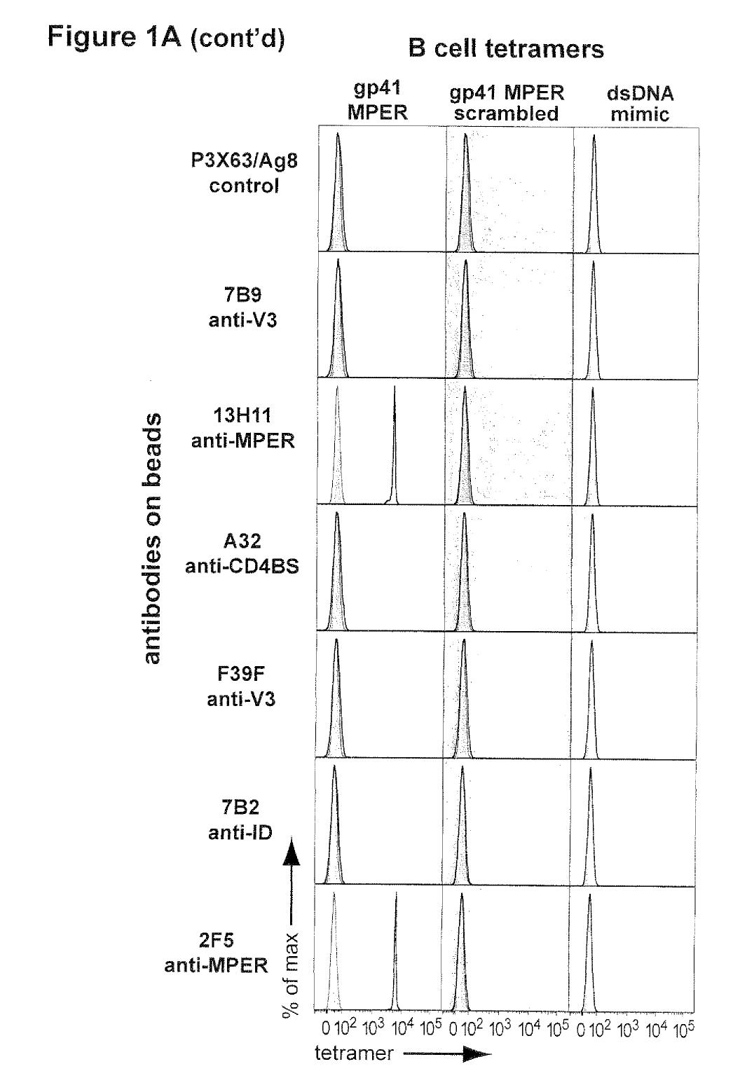

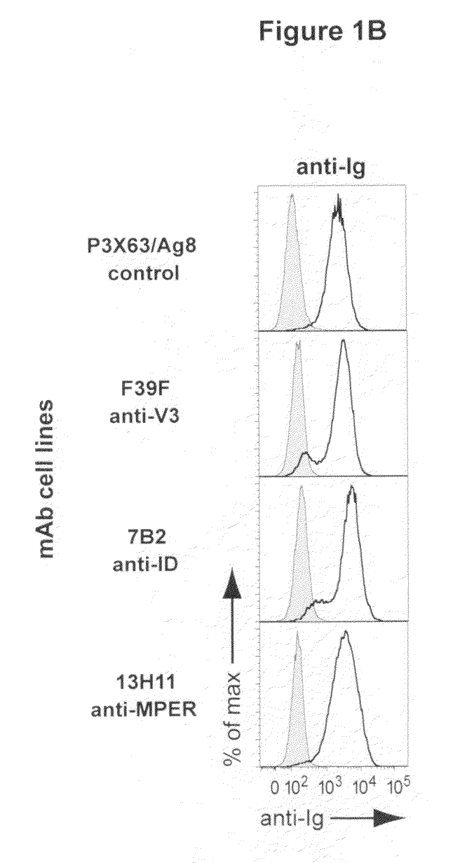

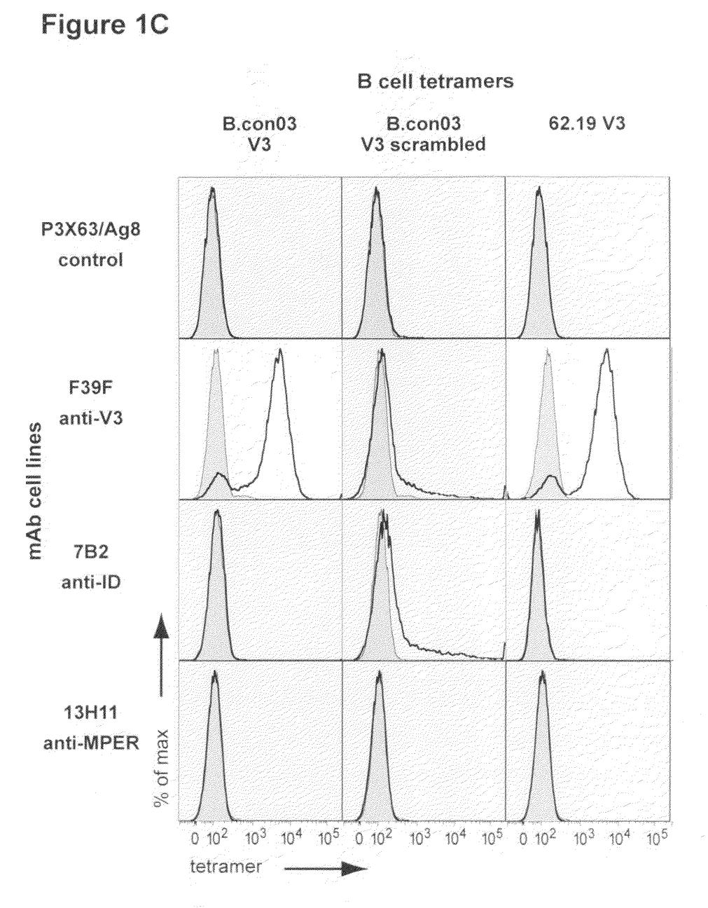

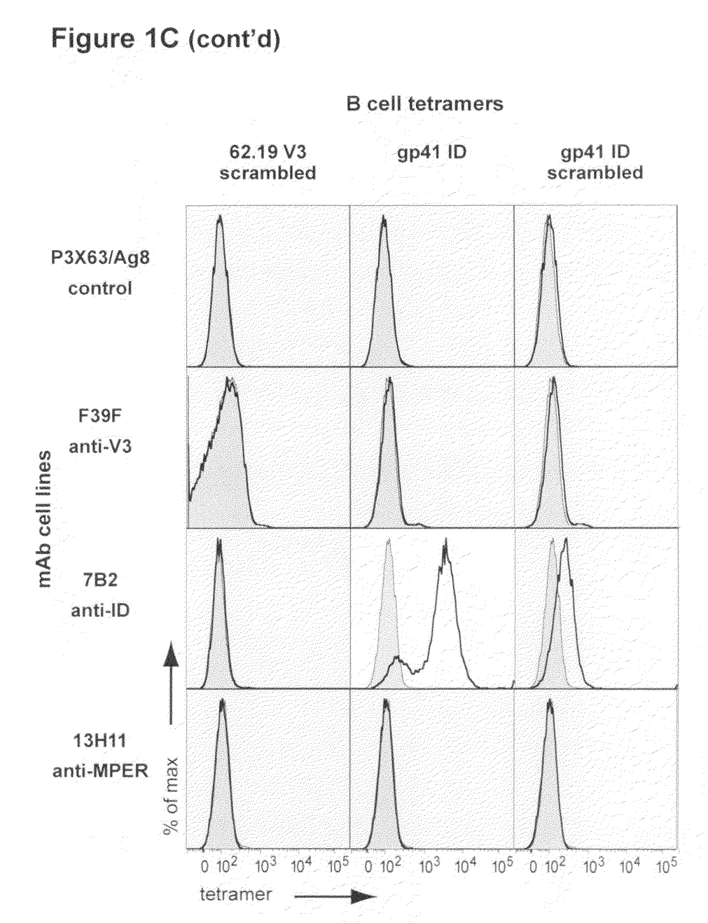

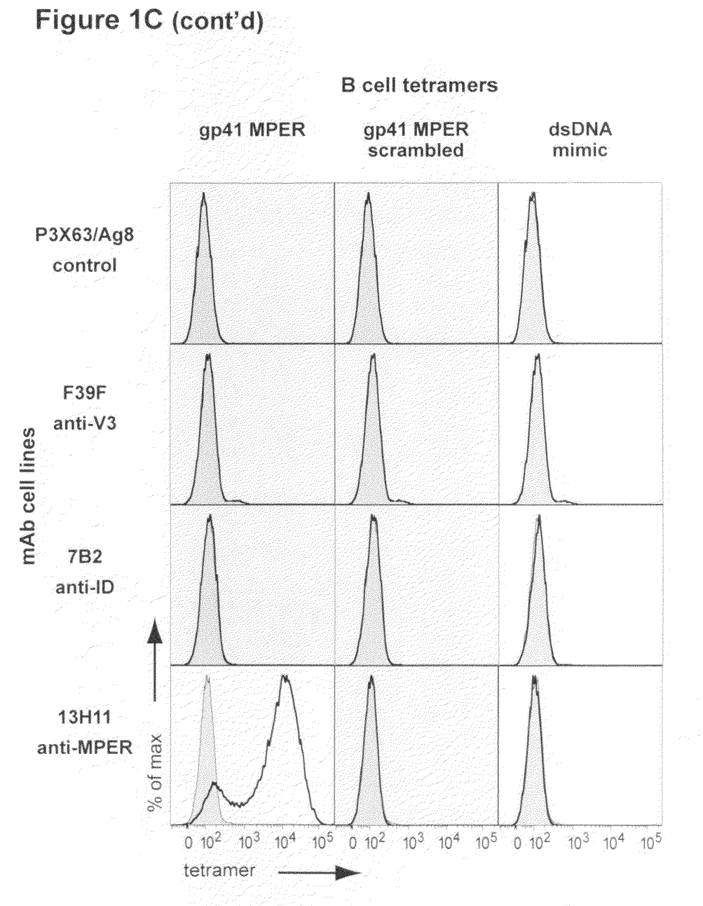

Tetramer specificity by reactivity with antibody-coated beads. Tetramer reactivity was next assayed with antibody-coated beads to determine tetramer specificity by flow cytometry. None of the tetramers bound to control mAbs P3X63/Ag8 or A32 on beads (FIG. 1A). The two gp120 V3 loop tetramers, B.con03 V3 and 62.19 V3, bound to both V3 loop mAbs 7B9 and F39F on beads. These data contrasted with the data obtained by SPR (Table 5) and suggests that immobilization of mAb 7B9 on the SPR chip altered its reactivity to the B.con03 V3 tetramer. The B.con03 V3 and the 62.19 V3 peptides differ in that 62.19 V3 is shorter and ends with the sequence ATE where B.con03 V3 has the sequence TTG. mAb 7B9 was derived from a mouse immunized with the shorter 62.19 V3 peptide and prior mapping studies of mAbs 7B9 and F39F showed a preference of 7B9 for the ATE sequence (see Table 6).

TABLE 6
Peptide mapping of anti-gp120 V3 mAbs.

mAb

| | | | |
|---|---|---|---|
| | | | P3X63/ |
| F39F | 7B9 | A32 | Ag8 |

| | |
|---|---|
| peptide sequence | OD @ 405 nm |

| | | | | |
|---|---|---|---|---|
| RPNNNTRKSIHIGPGRAFYATE | 0.956 | 0.350 | 0.068 | 0.065 |
| RPSNNTRKGIHLGPGRAIYATE | 0.131 | 0.474 | 0.139 | 0.121 |
| RPNNNTRKSIQIGPGRAFYTTG | 0.491 | 0.071 | 0.087 | 0.074 |
| RPNNNTRKSINIGPGRAFYTTG | 0.872 | 0.087 | 0.081 | 0.085 |

ELISA was performed at 1 μg/mL for F39F and A32 and at 0.063 μg/mL for 7B9 and P3X63/Ag8. Eight other peptides with sequences ending in TTG were also tested and were negative for all four mAbs.

The gp41 immunodominant region tetramer bound only to mAb 7B2 on beads; there was a higher background seen with the gp41 immunodominant scrambled tetramer on mAb 7B2, although the difference between the two tetramers was sufficient to distinguish positivity. The gp41 MPER tetramer bound to both the non-neutralizing mAb 13H11 and the neutralizing anti-gp41 mAb 2F5 on beads. None of the tetramers bound to a bead coated with an antibody for which they were not specific, and none of the scrambled tetramer controls showed significant binding to any antibody on beads. Finally, the dsDNA mimic tetramer did not bind to any of the antibody coated beads tested.

Monoclonal antibody cell lines show the specificity of the tetramer reagents. Monoclonal Ab secreting cell lines were next tested with anti-immunoglobulin (Ig) reagents in flow cytometry to determine the surface expression of Igs to demonstrate their usefulness as positive controls for B cell tetramer binding. Tested cell lines displayed a range of surface expression of Ig and four cell lines with high surface Ig levels were used for tetramer binding studies (FIG. 1B). Ig+ cell lines were incubated with tetramers and analyzed by flow cytometry (FIG. 1C). All tetramers were specific in that they each showed binding to the Ig+ cell lines expressing antibodies specific for those tetramer sequences. Cells with no surface Ig expression showed no binding of tetramers (data not shown). As with the mAb 7B2-coated beads, the gp41 immunodominant region scrambled tetramer did show higher background over unstained 7B2 cells. None of the tetramers bound to the control cell line P3X63/Ag8, and no cell line bound the dsDNA mimic tetramer.

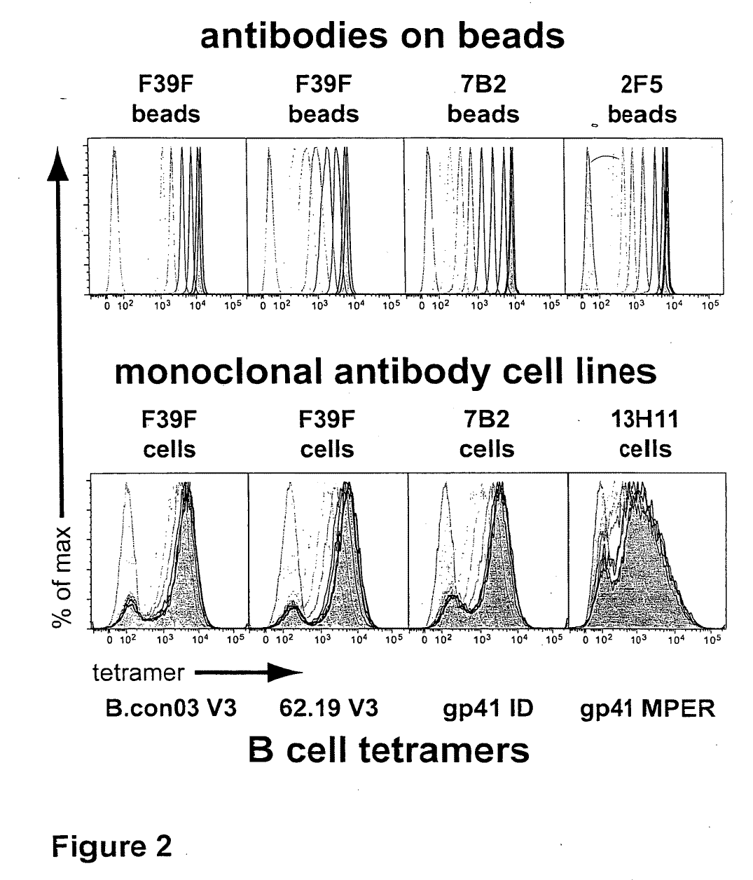

Titration of B cell tetramer reagents on antibody-coated beads and cell lines. On both antibody-coated beads and mAb cell lines, the tetramers bound with increasing signal as the concentration of tetramer was increased. In each case, tetramer binding reached saturation (FIG. 2). Similar experiments performed with beads or cell lines displaying antibodies that were not specific for the tetramer tested did not give signals above background (data not shown).

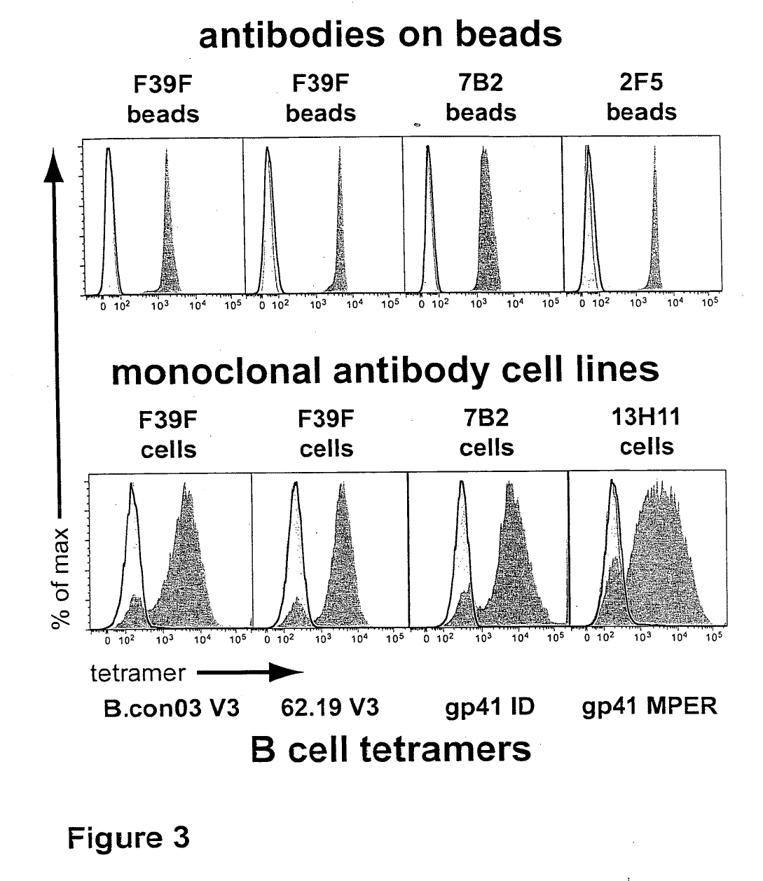

To further demonstrate the specificity of binding of the tetramers to beads and cell lines, antibody-coated beads or mAb expressing cell lines were incubated with APC-labeled tetramer either in the presence or absence of 10-fold excess unlabeled tetramer. In the presence of unlabeled tetramer, binding of APC-labeled tetramer was reduced to the level of the unstained controls (FIG. 3). Thus, using both antibody-coated beads and mAb cell lines, it has been demonstrated that the tetramers bind in a specific and saturable manner and is B cell receptor mediated.

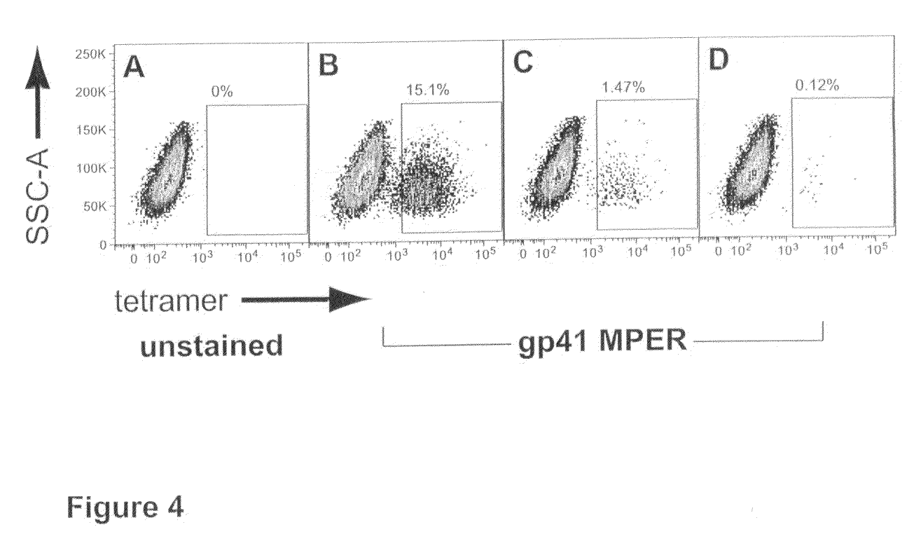

Detection of small populations of tetramer positive B cells. The minimum number of tetramer positive B cells detectable in a heterogeneous population of B cells was determined by assaying mixtures of cell lines with tetramers specific for the minority component. Shown in FIG. 4A is the unstained mixture of 85% anti-gp120 V3 mAb F39F cells and 15% anti-gp41 MPER 13H11 hybridoma cells as a negative control. FIG. 4B shows the same cell mixture incubated with the gp41 MPER tetramer showing that 15.1% of the cells stained with that tetramer. FIG. 4C shows the cell mixture from FIG. 4B diluted tenfold with additional anti-gp120 V3 mAb F39F cells resulting in a tenfold reduction of tetramer binding to 1.47%. FIG. 4D shows the cell mixture from FIG. 4C further diluted tenfold with anti-gp120 V3 mAb F39F cells resulting in 0.12% of the cells staining over background. These data show that decreasing numbers of B cell receptor positive cells reactive with tetramer are easily discriminated. Experiments performed with other combinations of control, gp120 V3-specific, and gp41 immunodominant-specific mAb cell lines and their respective tetramers gave similar results (data not shown).

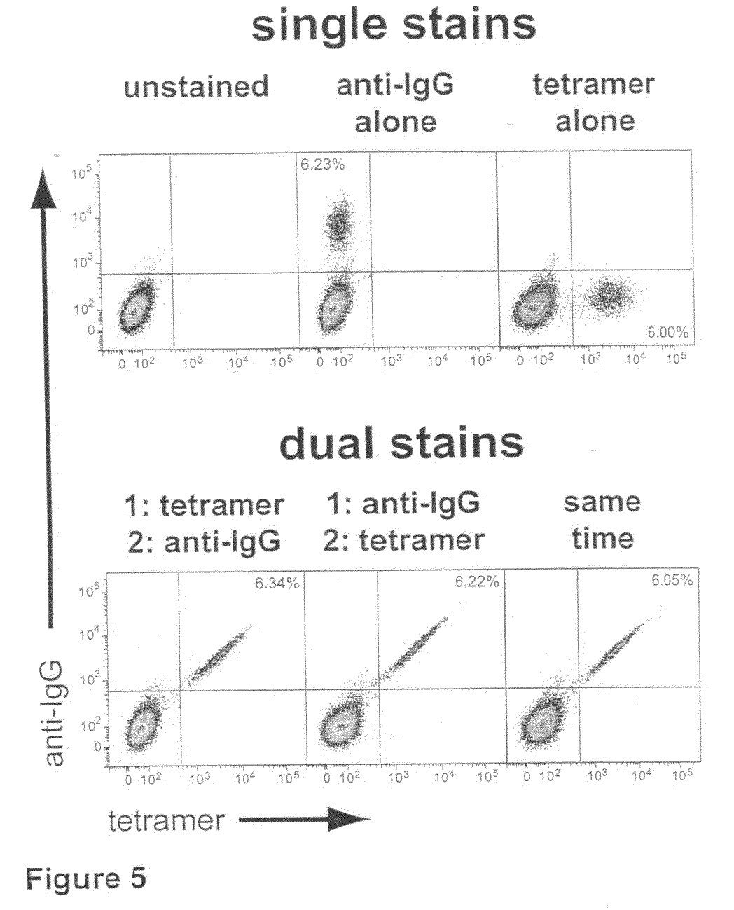

Lack of cross competition of anti-immunoglobulin reagents and tetramers. Because tetramers and anti-Ig reagents will both bind to B cell surface Ig receptors and because a key use of B cell tetramers will be to co-stain IgM, IgG, or IgA+ B cells with anti-Ig reagents and tetramers, there could be competition or hindrance of binding of one reagent by the other. FIG. 5 demonstrates that a PE labeled anti-IgG heavy chain reagent from BD Biosciences (Mountain View, Calif.) does not inhibit 62.19 V3 tetramer binding. The upper panels show a mixture of 6% human anti-gp120 V3 mAb F39F cells and 94% murine control P3X63/Ag8 cells unstained, stained with the anti-IgG reagent alone, or stained with 62.19 V3 tetramer alone. The bottom panels show double stains using the anti-IgG reagent and the 62.19 V3 tetramer showing that the percentage of positive cells is the same regardless of the order of addition of the anti-IgG reagent and tetramer. Experiments using a FITC labeled goat-anti-human IgG(H+L) reagent (Kirkegaard & Perry Laboratories, Inc., Gaithersburg, Md.) and gp41 immunodominant and MPER tetramers gave similar results (data not shown).

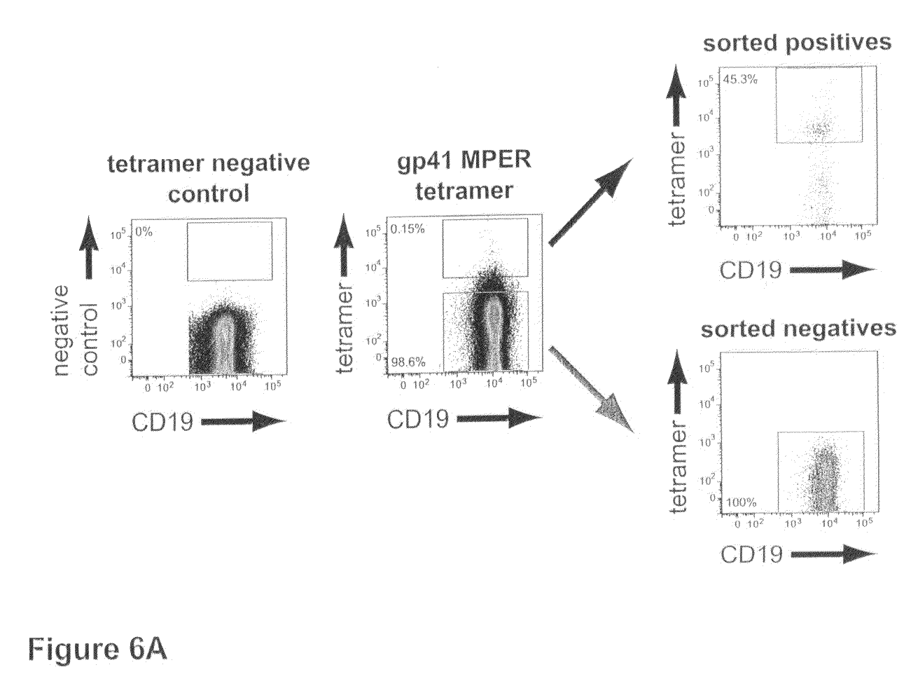

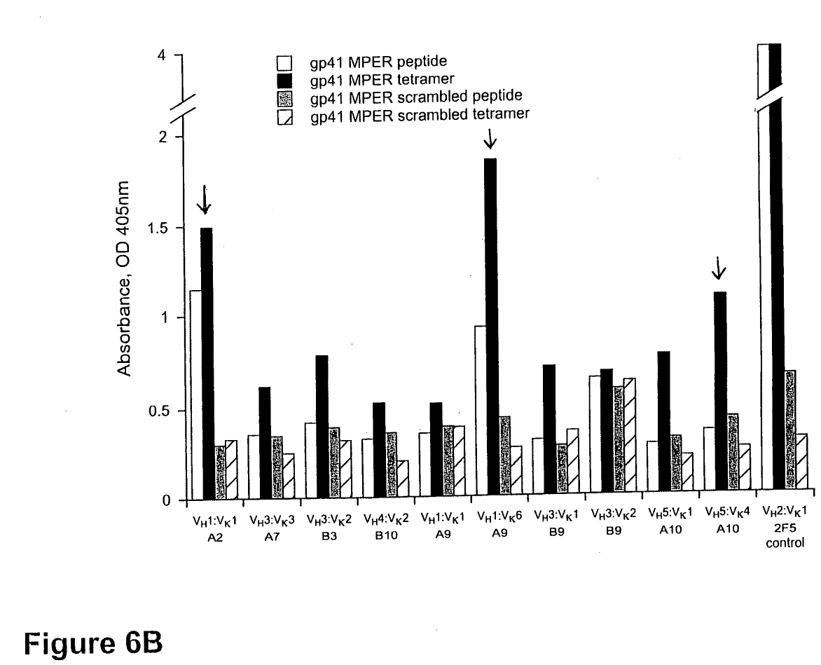

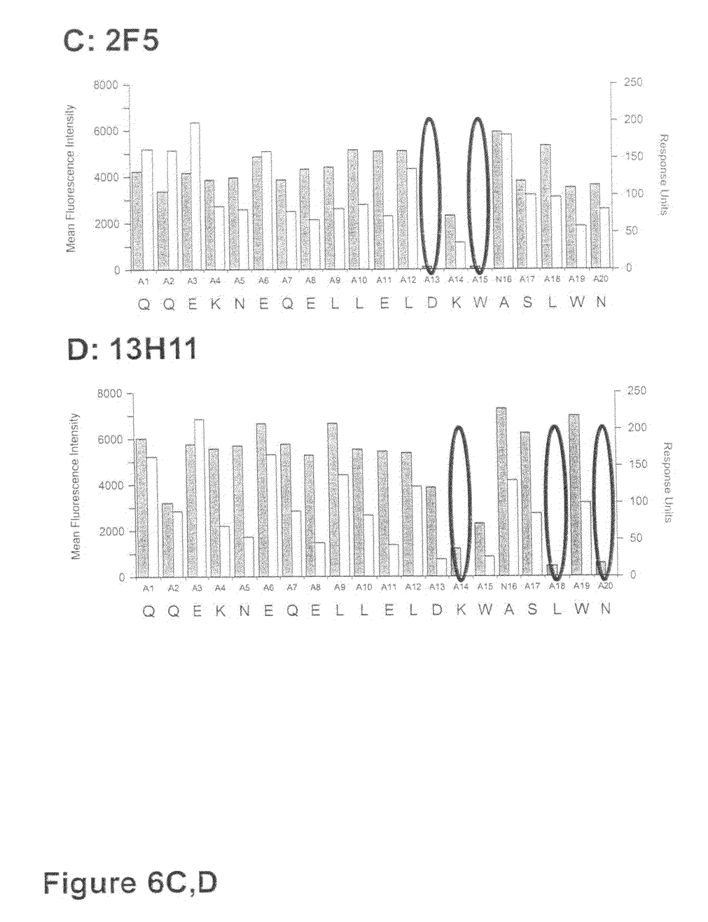

Sorting of tetramer binding B cells. PBMC obtained from six HIV-1 infected subjects were enriched by depletion of non-B cells and reacted with gp41 MPER tetramer and antibodies recognizing B cells, T cells, NK cells, and monocytes. Sorting was performed to place single gp41 MPER tetramer reactive B cells in 96-well plates. The left panel of FIG. 6A shows a negative control of CD3⁻ CD14⁻ CD16⁻ CD19⁺ B cells not stained with tetramer used to set sorting gates. The middle panel shows B cells stained with antibodies and gp41 MPER tetramer; the box gates shown were used to sort cells based on the presence or absence of tetramer binding. The right panels show sorted cells reanalyzed by flow cytometry to demonstrate the achieved purity. The gp41 MPER tetramer positive cells in this sample were enriched from 0.15% to 45.3% of CD19⁺ B cells. The tetramer negative population showed complete depletion of tetramer positive cells.

Amplification of V_{H} and V_{L} genes and production of antibodies from gp41 MPER tetramer positive sorted single cells. gp41 MPER tetramer positive B cells were sorted from a chronically HIV-1 infected patient as single cells into 96-well plates, RNA was isolated from these cells, and cDNA libraries were produced for each well. PCR primers specific for human variable heavy (V_{H}) and variable light (V_{L}) families were used to amplify and isolate immunoglobulin genes. cDNA prepared from a cell line expressing 2F5 mAb was used as a positive control. From 7 of 20 wells, both V_{H} and V_{L} genes were isolated (see Table 7). These genes were then cloned into expression vectors to produce a full length IgG heavy and kappa light chains. These expression vectors were then transiently co-transfected into 293T cells and secreted antibody from each pair tested by ELISA against gp41 MPER tetramer. Three of 10 expressed antibodies isolated from gp41 MPER tetramer positive B cells reacted with gp41 MPER tetramer but not with the scrambled gp41 MPER tetramer (FIG. 6B). A proportion of tetramer positive B cells were expected to be non-specific based on the percent of bare tetramer positive B cells. Therefore, the demonstration of 3 of 10 sorted gp41 MPER tetramer positive B cells reactive with gp41 MPER peptides confirms the utility and antigen-specificity of the gp41 MPER tetramers.

TABLE 7
Heavy and light chain pairs isolated from
sorted gp41 MPER tetramer positive B cells.

| single cell number | heavy chain | light chain | Ig isotype |
|---|---|---|---|
| A2 | V_{H}1 | V_{κ}1 | IgD |
| A7 | V_{H}3 | V_{κ}3 | IgM |
| B3 | V_{H}3 | V_{κ}2 | IgM |
| B10 | V_{H}4 | V_{κ}2 | IgM |
| A9 | V_{H}1 | V_{κ}1 | IgM |
| A9 | V_{H}1 | V_{κ}6 | IgM |
| B9 | V_{H}3 | V_{κ}1 | IgM |
| B9 | V_{H}3 | V_{κ}2 | IgM |
| A10 | V_{H}5 | V_{κ}1 | IgD |
| A10 | V_{H}5 | V_{κ}4 | IgD |

Development of gp41 MPER tetramers specific for neutralizing vs. non-neutralizing antibodies. The gp41 MPER tetramer bound both the non-neutralizing gp41 MPER mAb 13H11 and the neutralizing gp41 MPER mAb 2F5. Only rare HIV-1 infected subjects make 2F5-like gp41 MPER neutralizing antibodies, whereas it has been shown previously that over 80% of HIV-1 infected patients make non-neutralizing MPER antibodies (Alam et al, J. Virol. 82:115-125 (2008)). Therefore, the next question asked was whether gp41 MPER tetramers could be made that preferentially react with either of these two prototype antibodies that would make it possible to discriminate between neutralizing 2F5-like gp41 MPER antibodies and non-neutralizing 13H11-like MPER antibodies. To address this issue, a series of gp41 MPER tetramers were made with alanine substitutions along the primary sequence (see Table 8) and assayed for the binding of 13H11 or 2F5 by flow cytometry on antibody-coated beads and by SPR (FIGS. 6C and 6D). FIG. 6C demonstrates that the A13 and A15 gp41 MPER tetramer mutants both eliminated binding to 2F5 mAb. FIG. 6D demonstrates that, in contrast, the A14, A18, and A20 gp41 MPER tetramer mutants significantly reduced or eliminated binding to non-neutralizing mAb 13H11. Thus, tetramers made with the A13 or A15 mutant will bind B cells only with surface Ig similar to the non-neutralizing 13H11 antibody while tetramers made with the A14, A18, or A20 mutants will bind B cells only with surface Ig with specificities similar to the neutralizing 2F5 antibody.

TABLE 8

Amino acid sequences of alanine substituted
gp41 MPER tetramer peptides.

| Peptide | |
|---|---|
| name | Sequence |
| gP41 MPER | Q Q E K N E Q E L L E L D K W A S L W N |
| A1 | A Q E K N E Q E L L E L D K W A S L W N |
| A2 | Q A E K N E Q E L L E L D K W A S L W N |
| A3 | Q Q A K N E Q E L L E L D K W A S L W N |
| A4 | Q Q E A N E Q E L L E L D K W A S L W N |
| A5 | Q Q E K A E Q E L L E L D K W A S L W N |
| A6 | Q Q E K N A Q E L L E L D K W A S L W N |
| A7 | Q Q E K N E A E L L E L D K W A S L W N |
| A8 | Q Q E K N E Q A L L E L D K W A S L W N |
| A9 | Q Q E K N E Q E A L E L D K W A S L W N |
| A10 | Q Q E K N E Q E L A E L D K W A S L W N |
| A11 | Q Q E K N E Q E L L A L D K W A S L W N |
| A12 | Q Q E K N E Q E L L E A D K W A S L W N |
| A13 | Q Q E K N E Q E L L E L A K W A S L W N |
| A14 | Q Q E K N E Q E L L E L D A W A S L W N |
| A15 | Q Q E K N E Q E L L E L D K A A S L W N |
| N16 | Q Q E K N E Q E L L E L D K W N S L W N |
| A17 | Q Q E K N E Q E L L E L D K W A A L W N |
| A18 | Q Q E K N E Q E L L E L D K W A S A W N |
| A19 | Q Q E K N E Q E L L E L D K W A S L A N |
| A20 | Q Q E K N E Q E L L E L D K W A S L W A |

Summarizing, the development and quality control of a series of B cell tetramers specific for linear epitopes of HIV-1 is described above and the utility of a B cell tetramer specific for the HIV-1 gp41 MPER to identify and sort small populations of tetramer reactive CD19⁺ B cells in HIV-1 infected subjects is shown.

A number of investigators have previously shown that detection of antigen specific B cells was possible in certain contexts. Cells from mice immunized with protein antigens have been sorted using fluorescently labeled versions of those proteins including keyhole limpet hemocyanin (Julius et al, Proc. Natl. Acad. Sci. USA 69:1934-1938 (1972)), ovalbumin and Helix pomatia hemocyanin (Hoven et al, Journal of Immunological Methods 117:275-284 91989)). Both naïve and immunized mice have been probed for hapten-specific B cells using trinitrophenyl (Greenstein et al, J. Immunol. 124:1472-1481 (1980)) and (4-hydroxy-3-nitrophenyl)acetyl (Lalor et al, Eur. J. Immunol. 22:3001-3011 (1992), McHeyzer-Williams et al, J. Exp. Med. 191:1149-1166 (2000), McHeyzer-Williams et al, Nature 350:502-505 (1991)) derivatized fluorescently labeled proteins. Julius et al (Eur. J. Immunol. 6:288-292 (1976) demonstrated the use of two different antigens for sorting and characterizing the monospecificity of surface Ig on B cells. Hayakawa et al. (Proc. Natl. Acad,. Sci. USA 84:1379-1383 (1987)) sorted phycoerythrin-binding splenocytes from mice immunized with phycoerythrin. A whole virion approach was described by Doucett et al (Journal of Immunological Methods 303:40-52 (2005)) to identify and purify populations of B cells reactive with whole influenza virus as well as with biotinylated hemagglutinin. Newman et al (Journal of Immunological Methods 272:177-187 (2003)) described the preparation and use of a B cell tetramer using a biotinylated peptide previously shown to be a potent inhibitor of a pathogenic dsDNA antibody. Using this reagent, they were able to demonstrate a population of cells induced by immunization and to characterize them by flow cytometry.

As a result of the foregoing studies, a panel of reagents is provided suitable for exploring the repertoire of epitope specific antibody responses to a single protein, and bare and scrambled tetramers are described that make it possible to rigidly control for specificity. Bare tetramer, consisting of free biotin reacted with APC-labeled streptavidin, will control for streptavidin and APC binding B cells, while scrambled tetramers, consisting of primary sequence scrambled peptides for each of the HIV-1 epitope sequences, will control for non-specific charge or hydrophobicity based binding. The diversity of the B cell repertoire is such that B cells reactive with bare or scrambled tetramers that are unrelated to B cells reactive with native sequence tetramers are expected.

Using these reagents, antibodies have been produced from cells reactive with gp41 MPER tetramer and their ability to bind to tetramer has been demonstrated in an ELISA assay. The ability to perform bulk and single cell characterization for a range of epitopes for HIV-1 greatly expands the capacity to dissect the B cell response to this virus. With this panel of well characterized reagents specific for a variety of epitopes of HIV-1, it is possible to probe, at a cellular level, the differences between commonly made responses, such as the gp41 immunodominant region, and rare responses, such as neutralizing gp41 MPER antibodies. The scarcity of such broadly neutralizing antibodies and the rarity of such antibodies in both immunized and infected subjects has been a major hindrance for the field. These reagents make it possible to probe the basis for the rarity of these antibodies and also offer a way to investigate the immune response to infection and immunization.

The panel of alanine substituted gp41 MPER tetramers offer particular promise for this work. The A13 and A15 mutants demonstrate minimal reactivity with 2F5 consistent with the results from other mapping studies (Zwick et al, J. Virol. 79:1252-1261 (2005)). The A14, A18, and A20 mutants are of even greater interest, as they appear to be specific for the broadly neutralizing gp41 MPER mAb 2F5 and to have little binding to the non-neutralizing gp41 MPER mAb 13H11. As such, this panel of reagents can be used to characterize B cells with surface Ig reactive with each kind of tetramer and thus allow for comparison of cells with those differing specificities to determine if there is an immunological basis for the relative rarity of broadly neutralizing 2F5-like antibodies. Furthermore, these reagents can be used to study vaccine candidates and immunization strategies to determine whether they stimulate the desired response.

It has been shown that these reagents can identify a small population of cells in a clinical sample and that these cells can be further sorted into enriched and depleted populations or into single cell preparations. Given the transient period of time that antigen specific B cells may be present in peripheral blood and the scarcity of such cells (Blink et al, J. Exp. Med. 201:545-554 (2005)) the ability to sort and enrich these populations makes possible more intense investigation of these populations.

### Example 2

The goal of the study described below was to examine the interactions of B cells with the 2F5 MPER epitope, compared to other HIV-1 epitopes. Murine B cells were selected for study, since the problem of hampered 2F5 Ab responses is not species-specific and complex studies of B cell genetics and ontogeny can be simplified in the mouse, where congenic strains and developmental B cell subsets are well-defined. Using recently developed B cell HIV-1 epitope-specific tetramer reagents, two distinct types of interactions were demonstrated to exist between murine B cell receptors and the MPER 2F5 epitope: 1) a superantigen-like interaction involving allotype-restricted binding of B cells to MPER residues outside the neutralization-sensitive motif, and 2) developmentally-regulated, allotype-independent binding, consistent with developmentally-regulated tolerization of the 2F5 nAb B cell repertoire. Overall, the findings not only support the idea that antigen-specific tolerance mechanisms suppress the bnAb B cell repertoire in the bone marrow, but suggest that additional, antigen-independent mechanisms may also immunoregulate B cell responses to the MPER. The residues involved in the latter mechanism may play a role in hampering a 2F5 bnAb response by “unfocusing” an antigen-specific response away from B cell interactions with neutralization-specific residues in the MPER. Most importantly, this B cell tetramer technology provides both for design of antigen-specific reagents to purify and clone antibodies from specific B cells, as well as design of vaccines to induce desired antibodies.

### Experimental Details

B cell tetramer synthesis and validation. N-biotinylated, linker/spacer-containing peptides used were as follows: gp41 2F5 MPER (biotin-GGGQQEKNEQELLELDKWASLWN; gp41 Env 659-678, containing the gp41 MPER 2F5 nominal epitope), scr. gp41 2F5 MPER (biotin-GGGNKEQDQAEESLQLWEKLNWL; a scrambled version of gp41 2F5 MPER), gp41 MPER alanine mutants A1-A15 and A17-A20 (with alanine substitutions in sequential sites across the wild type gp41 2F5 MPER epitope), the asparagine mutant N16 (with an asparagine substitution at the 16^{th} position in the gp41 2F5 MPER epitope), gp41 ID (biotin-GGGKQLQARVLAVERYLKDQQLLGIWGCSGKLICTTAV; gp41 Env 581-615, containing the gp41 immunodominant region epitope), scr. gp41 ID (biotin-GGGQLDSIQKEVYQLGRQLVWCTARLATKLVGKACGIL; a scrambled version of gp41 ID), 62.19 V3 (biotin-GGGTRPNNNTRKSIHIGPGRAFYATE; gp120 Env 306-328, containing a V3 loop epitope of gp120), scr. 62.19 V3 (biotin-GGGNPATISRTIGHNFRYTPAREKNG; a scrambled version of 62.19 V3), B.con V3 (biotin-GGGTRPNNNTRKSIHIG PGRAFYTTGEIIGDIRQAH; gp120 Env 306-328, containing another V3 loop epitope), scr. B.con V3 (biotin-GGGITIDNIGHHNRITFRAANTRPISGQRGEYPGKT, a scrambled version of B. con V3), and dsDNA mimic (biotin-GGGGGDWEYSVWLSN, containing a double-stranded DNA mimetope). Peptides were synthesized and purified using reverse-phase HPLC by Primm Biotechnology. Purity was confirmed by mass spectrophotometric analysis. To produce tetramerized forms of each of these peptides, 200 μM peptide and 6 μM allophycocyanin (APC)-labeled streptavadin (SA) were combined at equal volumes and mixtures were incubated at 4° C. for a minimum of 4 hours. Subsequently, unbound peptide was removed from peptide-APC complexes by centrifugal filtration using an Amicon Centriprep YM30 column (Millipore Corporation). Peptide complexes with unlabeled SA were similarly generated and used in excess in cold competition assays. Purified tetramer preparations were determined using the Micro BSA protein assay kit (Pierce Biotechnology). For use in flow cytometry, tetramer specificity was verified using either beads coated with antibodies against epitopes of interest or with monoclonal cell lines expressing those antibodies. Additionally, each tetramer was titrated in order to determine the optimal concentration range for staining (see FIG. 2).

Mice. Female C57BL/6, BALB/c, B6Igh^{a}, and CB17 (BALBc Igh^{b}) inbred mouse strains (8-12 weeks of age) were purchased from Charles River Laboratories. All mice were housed in the Duke University Animal Facility in a pathogen-free environment with 12 hour light/dark cycles at 20-25° C. under AALAC guidelines and in accordance with all Institutional Animal Care and Use Committee and Duke University Institutional Biosafety Committee-approved animal protocols.

Peptide immunogen formulation, dose, route of immunization, and immunization schedule. The peptide immunogen used was a gp41 heptad repeat-2 (HR2) DP 1 78Q16L peptide (YTSLIHSLIEESQNQQEKNEQELLELDKWASLWNWF), synthesized and purified from Primm, and verified to be >95% purified as determined by mass spectrometry. Lyophilized peptide immunogens stored at 4° C. (Primm Biotechnology) were reconstituted by dissolving in DMSO at 8 mg/ml and bringing up to 1 mg/ml in saline. Adjuvanting was done by dissolving mouse oCpG, (5′-TCCATGACGTTCCTGACGTT-3′; Midland Certified Reagent Company) in saline at 1 mg/ml and first mixing with Oil/water Emulsigen (MVP laboratories) in saline, then with peptide, such as to create a final mixture of 50 μg/ml oCpG, 125 μg/ml peptide, 10% Emulsigen (for i.p. immunizations) and 200 μg/ml oCpG, 500 μg/ml peptide, 10% Emulsigen (for i.m. immunizations). Immunization primes and boosts were all done at a single site intraperitoneally using a volume of 200 μl of the above formulation, such that mice in all instances were injected with 25 μg peptide and 10 μg oCpG. Mice were primed for 10 days and then boosted four times, every 14 days.

Cell isolation, serum collection, and PBMC isolations. Heparinized or non-heparanized blood was collected sub-mandibularly 10 days after immunizations. For preparation of peripheral blood lymphocytes for B cell tetramer analysis, PBMC were isolated from blood collected in heparinized tubes (0.2 ml) using Lympholyte M (Cederlane). For Luminex and ELISA assays, sera was isolated from non-heparanized blood and stored at −20° C. until use. Spleen, bone marrow (BM), and peritoneal cavity (PerC) cells were also collected. For most immunizations, tissues were harvested after the 4^{th} boost, except for kinetic studies, in which tissues from mice were harvested after 1, 3 or 7 days after a single immunization. For BM preparations, femora and tibiae were removed, aspirated with tissue medium using 23 gauge needles, filtered with 70 μm cell strainers (BD labware), and RBC-depleted using Tris-buffered ammonium chloride lysis. For splenocyte preparation, spleens were harvested and dissociated into single-cell suspensions either by passage through 70 μm cell strainers in tissue medium or by using frosted glass slides, and blood erythrocytes were depleted with ammonium chloride lysis. For isolation of peritoneal cells, peritoneal cavities of sacrificed mice were lavaged by injection with 10 ml of PBS, followed by gentle massage of the abdomen, and withdrawal of the peritoneal exudates. BM, splenic, and peritoneal single cell suspensions were centrifuged at 1500 rpm for 5 min and resuspended in 5, 10, and 1 ml of tissue media, respectively, for cell counts and immunofluorescence staining. Cell counts were performed in duplicate on a Coulter ZI Dual Threshold Cell Counter (Coulter).

Antibodies and flow cytometry. Immunofluorescent staining of single cell suspensions (≧2×10⁶) was conducted using pre-mixed combinations of fluorochrome-labeled antibodies and APC-conjugated B cell tetramers at empirically-determined optimal concentrations. All antibodies were from BD unless otherwise stated. Primary antibodies used for phenotyping bone marrow, spleen, and peritoneal B cells included PerCP anti-B220 (clone RA3-6B2), PE anti-kappa (clone 187.1), FITC anti-IgD (clone 11-26c.2a), FITC, PE or PE-Cy7 anti-IgM (clone R6-60.2), PE anti-CD5 (clone 53-7.3), FITC anti-CD11b (clone M1/70), PE-Cy7 anti-CD19 (clone 1D3), FITC anti-CD21 (clone 7G6), PE-Cy7 anti-CD23 (eBiosciences; clone BSB4), PE anti-CD43 (clone S7), and PE-Cy7 anti-CD93 (eBiosciences; clone AA4.1). Other reagents included biotinylated antibodies against dump channel markers: Thy1 (Ox7; Abcam), F4/80 (BM8; Abcam), CD11c (clone HL3), Gr-1 (clone RB6-8C5), TER-119, and NK-1.1 (clone PK136), secondary staining SA-Texas Red conjugate; v-amine live/dead violet dye (Molecular Probes), and Fc block (anti-CD16/32; clone 2.4G2).

Staining was conducted at 4° C. in several sequential steps in FACS staining buffer (1×PBS with 2% FCS, 0.1% NaN₃), washing twice between steps. First, cells were incubated with v-amine live/dead dye for 15 min. Next, cells were stained with APC-labeled tetramers for 30 min. Where applicable, this was preceded by addition of cold tetramer at 10 fold molar excess for 1 h. Finally, cells were stained with Fc block for 30 min, followed by incubation with combinations of primary antibody cocktails for 30 min, and secondary staining with SA-Texas Red for 30 min.

Data were acquired using an LSRII flow cytometer and Cell Quest software (BD Immunocytometry systems). A minimum of 250000 events were acquired per sample. FACS Analysis was performed using FlowJo software (Tree Star). For analysis, single cells were positively gated based on their forward scatter height vs. forward scatter area and side scatter height vs. side scatter area profiles, lymphocytes were then positively gated based on their forward and side scatter profile (live lymphocyte gate), residual dead cells were excluded with negative gating on v-amine live/dead dye, and all irrelevant non-B cells were excluded with negative gating on dump markers Thy1, F4/80, CD11c, Gr-1, TER-119, and NK-1.1.

B cell purification, BCR internalization assay, and microscopy of BCR cap formation. Splenic B cells were purified by negative depletion of CD43-expressing non B cells with anti-mouse CD43 MACS beads (Miltenyi Biotechnology) resulting in fractions >90% B cells.

For analysis of BCR internalization by flow cytometry, purified B cells were pre-incubated on ice for 30 min with either a 95:5 unlabeled/R-PE-labeled mixture of 10 μg/ml goat anti-mouse-IgM+IgG (H+L) F(ab)₂ fragments (Jackson ImmunoResearch laboratories). To remove excess reagent, cells were then washed, resuspended in DMEM 10% FCS, and stimulated by warming to 37° C. (or at 4° C. as a control) for the indicated time points. Cells were then fixed immediately (for time zero) or at the indicated time points with 1% methanol-free formaldehyde PBS solution, stained with PerCP-B220 and either APC-labeled gp41 MPER or scrambled gp41 MPER tetramers, and surface-bound anti-IgM+IgG and tetramers were determined by flow cytometric detection. Percent internalization for tetramers was calculated on gp41 MPER+B220+ subsets (the gp41 MPER gate set based on the FMO control) by the formula [% gp41 MPER (Tₒ)−% gp41 MPER (Tₙ)]/% Sp62 (Tₒ)×100. In other experiments, cells were pre-incubated with either 5 mg/ml mannan, 125 mM α-methyl-mannopyranoside (Sigma-Aldrich), or 10 μg/ml unlabeled goat anti-mouse-IgM+IgG (H+L) F(ab)₂ fragments, prior to staining with APC-labeled tetramers.

To visualize specifically capped BCR complexes, naïve and DP178/T20-immunized splenic cells were incubated on ice for 30 min with Alexa488-labeled wheat germ agglutinin (Invitrogen) as a general surface marker) and 10 μg/ml R-PE-labeled goat anti-mouse IgM+IgG (H+L) F(ab)₂ and APC-labeled gp41 MPER. After extensive washing, cells were stimulated for the indicated time points. Images were acquired with a Nikon TE2000-E2 inverted microscope employing a 40× magnification lens from a CoolSNAPPHQ2 monochrome camera.

ELISA assays. For determination of 2F5 antibody titers, ELISA was performed by coating high-binding microtiter plates (Easywash, Costar 3369) with 2F5 nominal peptide (0.2 μg/well) in coating buffer (0.1 M sodium bicarbonate) at room temperature for 2 h and then at 4° C. overnight. Plates were then blocked with Super Block reagent (4% Whey, 15% goat serum, 0.05% Tween-20, 0.05% NaN₃), incubated with serum diluted at serial concentrations, then with a solution of 1 μg/ml alkaline phosphatase-conjugated goat anti-mouse IgG, all three steps done for 1 h at room temperature and using Super Block as diluent. Finally, plates were developed by incubating with 1 mg/ml substrate (p-nitrophenyl phosphate in 10 Mm MgCl₂ and 50 Mm NaHCO₃) in the dark for 45 min. Optical density was read at 405 nm with a BioRad 680 microplate reader. Plates were washed with PBS-0.1% Tween between all steps.

Purification of tetramer-reactive B cells and qPCR analysis of V_{H} family usage for V_{H}-J_{H}1 rearrangements. For sorting of gp41 MPER tetramer-binding B cells, single cell splenic suspensions were obtained from 10 naïve Balb/c females (approximately 8 weeks old), pooled, and purified by negative depletion of CD43-positive non B cells with anti-mouse CD43 MACS beads (Miltenyi Biotechnology). The resulting B cell enriched fraction was stained by flow cytometry as follows: non-viable cells were labeled using Live/Dead Fixable Violet Dead Cell Stain Kit (Invitrogen) for 15 minutes on ice, then washed with 1×PBS to remove excess stain. Next, cells were stained with either APC-labeled gp41 2F5 MPER tetramer or APC-labeled scrambled gp41 2F5 MPER tetramer for 30 minutes on ice, and then washed with staining buffer (PBS+3% FBS+0.1% NaN₃) to remove excess tetramer. Finally, cells were stained with PerCP-B220 and PE-Cy7-CD19 for 30 minutes on ice and washed with staining buffer to remove excess antibody. Cells were immediately sorted into B220⁺ CD19⁺ gp41 2F5 MPER⁺ and B220⁺ CD19⁺ gp41 2F5 MPER-negative fractions using a BDFACS Aria (Becton Dickinson). Sorting gates for the gp41 2F5 MPER tetramer⁺ fractions were set using the FMO control (i.e., staining without tetramer) as baseline i.e. include all events greater than those observed in the FMO gate, whereas gp41 2F5 MPER tetramer⁻ fractions were collected using a subset gate with several fold lower MFI than the total FMO control population.

Purity of sorted populations was verified by a 1000-event post-sort step. Immediately after sorting, cells were spun down, resuspended in lysis buffer, and incubated at 56° C. overnight. Genomic DNA was extracted using common phenol extraction techniques, precipitated using ethanol, and resuspended in molecular biology grade water (Invitrogen). Genomic DNA preparations were quantitated using a Nanoprop ND-1000 spectrophotometer (Thermo Fisher Scientific).

For analysis of V_{H} usage using V_{H} family-specific primers, qPCR amplification was performed using SYBR Green PCR Master Mix (Applied Biosystems) according to manufacturer's instructions and 25 ng gDNA using the following V_{H} family-specific primers (Invitrogen): V_{H}1 J558.1:AGA TAT CCT GCA AGG CTT CT; V_{H}1-2: TCT AGA ATT CAG GTC CAA CTG CAG CA; V_{H}3-1: GAT GTG CAG CTT CAG GAG TCA; V_{H}14-1: GAG GTT CAG CTG CAG CAG TCT; and J_{H}1: CCC GTT TCA GAA TGG AAT GTG C. CD14 was used to normalize utilization: 5′: GCT CAA ACT TTC AGA ATC TAC CGA C; and 3′: AGT CAG TTC GTG GAG GCC GGA AAT C. Initial denaturing was done at 95° C. for 10 min, denaturing was done at 95° C. for 10 sec, annealing and extension were done at 60° C. for 45 sec using 40 cycles with data collection and real-time analysis functions enabled. Final denaturing was done at 95° C. for 1 min, followed by annealing and extension at 55° C. for 70 sec. Amplifications were performed using an iCycler thermocycler and qPCR measurements were conducted using iQ5 Optical System Software v2.0 (BioRad). Relative V_{H} expression was determined using the cycle threshold counts for V_{H} families, which were normalized for the amount of input gDNA to cycle threshold counts for CD14 using the following equation: 2^{-(VH cycle threshold value-CD14 cycle threshold value)}.

Graphing was performed using Microsoft Excel.

Surface plasmon resonance (SPR) measurements. All SPR measurements were conducted on a BIAcore 3000 instrument and data analyses were performed using the BIAevaluation 4.1 software (BIAcore), as previously described (Alam et al, J. Immunol. 178(7):4424-4435 (2007)).

### Results

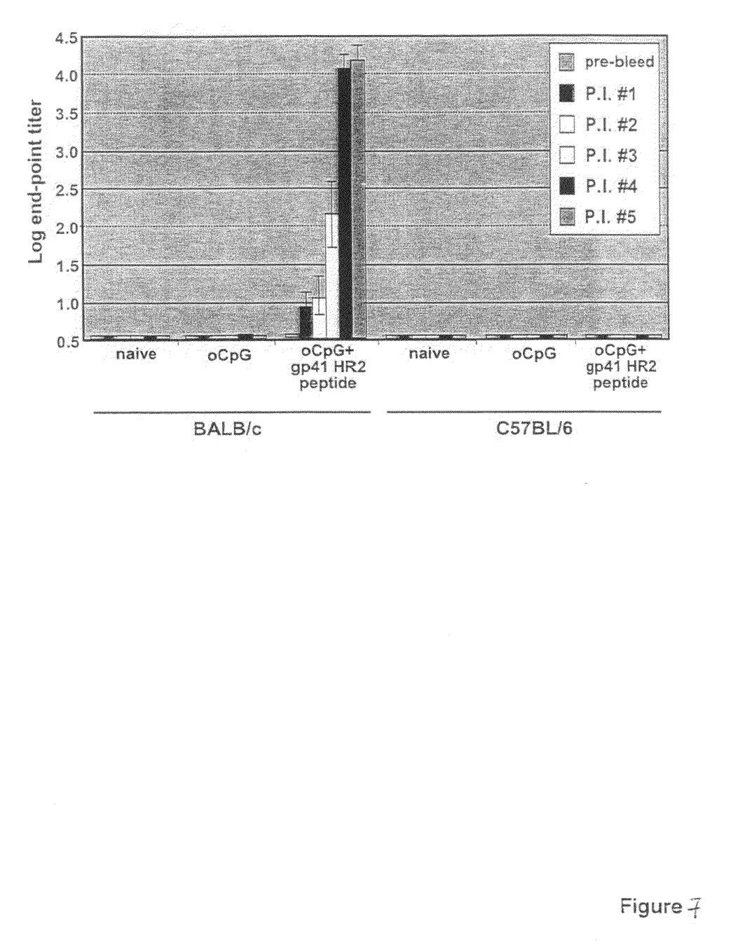

gp41 HR2 peptide immunization elicits a strain-specific anti-2F5 Ab response. Since no broadly neutralizing Ab responses can be elicited in any experimental animal model with any current HIV-1 immunogen, the rationale was to further characterize murine non-neutralizing Ab responses, as well as the B cell populations present in mice capable of eliciting such responses, with the idea that this may be the most practical animal model to gain some immunological and/or genetic insights into why neutralizing Ab responses are unattainable. In this context, it had been shown previously that immunization of BALB/c mice five times every two weeks with DP178Q16L/T20 (a peptide spanning the HR2 region of the MPER and containing the 2F5 epitope), when formulated in Emulsigen+oCpGs, generates a robust non-neutralizing cross-reactive anti-2F5-like responses (detectable by the third immunizations and peaking after the fourth immunizations) as assayed by ELISA for serum Ab titers to the 2F5 epitope 10 days after each immunization (Verkoczy and Haynes, unpublished results). In the further characterizations of parameters required for eliciting these non-neutralizing 2F5-specific Ab responses, i.e., varying adjuvanting, immunogen, route of immunization, mouse strain, etc., an unexpected finding was encountered upon performing parallel immunizations with the DP178Q16L peptide in BALB/c and C57BL/6 strains: while robust anti-2F5 serum Ab responses could be measured in DP178-immunized BALB/c mice, no appreciable anti-2F5 serum Ab titers could be detected at any point over the course of T20/DP178 immunizations in C57BL/6 mice (FIG. 7).

The gp41 2F5 MPER epitope specifically interacts with a large fraction of the total naïve splenic B cell population in a strain-specific manner. The strain-dependent difference in the non-neutralizing 2F5-specific Ab response suggested that there may be differential responses to, and interactions with, the 2F5 MPER epitope by B cell populations from C57BL/6 and BALB/c mice. To begin testing this possibility, an APC-labeled gp41 2F5 MPER epitope-specific tetramer reagent was used to detect total splenic B cell populations from unimmunized BALB/c and C57BL/6 mice by flow cytometry. gp41 2F5 MPER epitope-specific tetramers comprise one of a series of HIV-1 epitope-bearing tetramers recently developed for detecting antigen-specific B cells in surface staining assays (see Example 1). These reagents have been validated for binding specificity and sensitivity, using HIV-1 epitope-coated beads or HIV-1 epitope-specific monoclonal antibody-producing cell lines (as positive controls), and using scrambled control tetramer reagents or pre-incubating cells with unlabeled tetramers in molar excess (as negative controls).

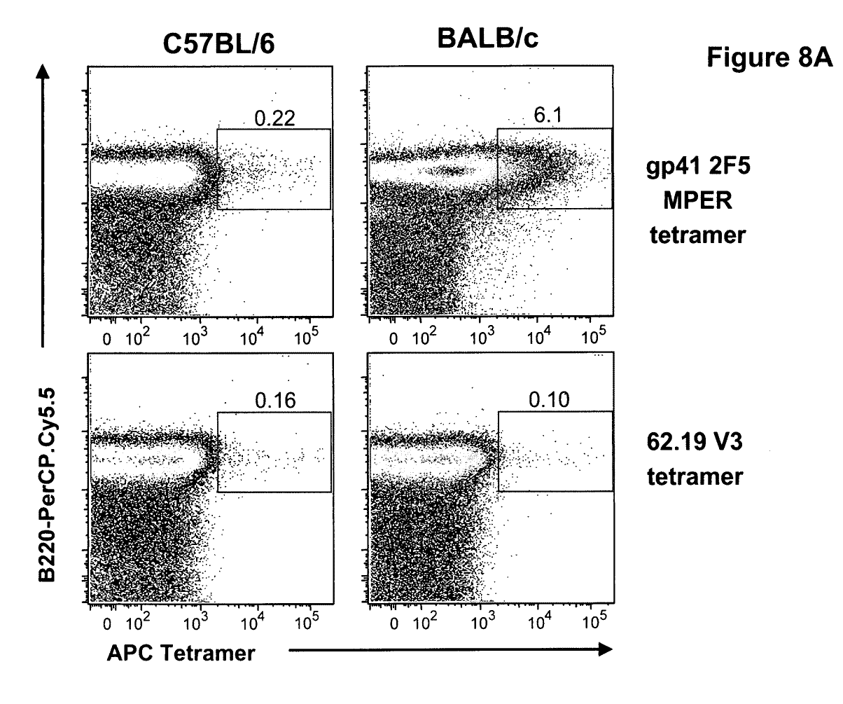

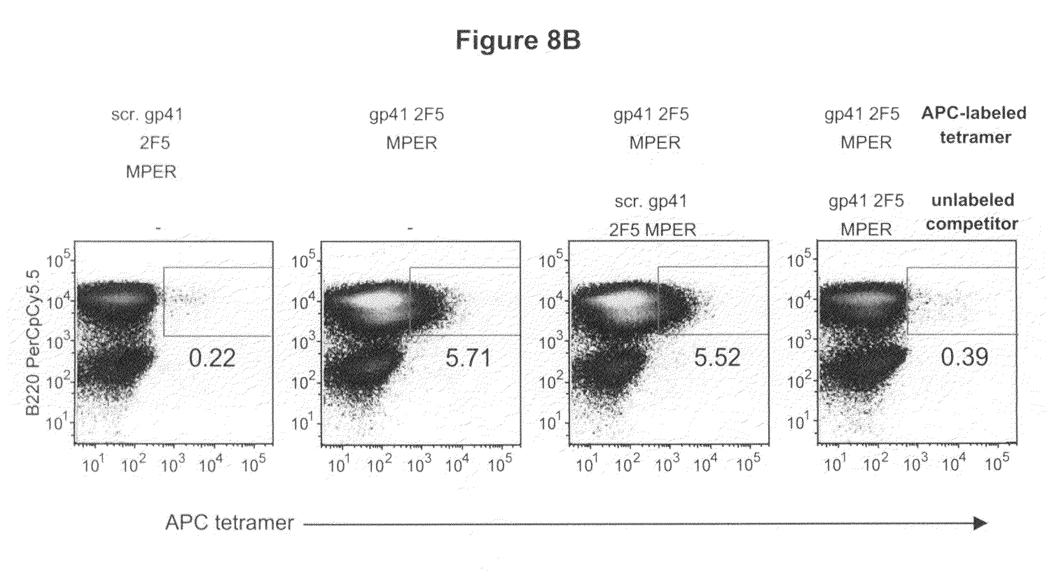

Strikingly, a significant fraction (>5%) of the total naïve splenic BALB/c B cell repertoire was found to bind the gp41 2F5 MPER-specific tetramer (FIG. 8A, top right panel), in contrast to negligible staining (<0.3%) of the total naïve splenic C57BL/6 B cell repertoire with gp41 2F5 MPER (FIG. 8A, top left panel). Additionally, staining with another HIV epitope-specific tetramer, 62.19 V3, used as a control, showed minimal binding in both strains of mice (FIG. 8A, lower panels).

To exclude the possibility that this high frequency interaction between the total B cell population and the 2F5 MPER epitope in naïve BALB/c mice was attributable to spurious binding, a series of experiments were conducted. First, an assessment was made as to whether the unusual charge of the MPER epitope played a role by staining B cells with a scrambled gp41 2F5 MPER tetramer (which contains the same composition of amino acids in a random order). Negligible binding was observed, ruling out cross-reactive, non-epitope specific binding (FIG. 8B). Splenocytes were also pre-incubated in medium containing a 10-fold molar excess of unlabeled gp41 2F5 MPER or gp41 2F5 MPER scrambled control tetramers for 30 min. on ice prior to labeling with APC-conjugated SP62-tetramers (FIG. 8B). Within the B220⁺ compartments, pre-incubation with the homologous gp41 MPER unlabeled tetramer (but not the scrambled control) inhibited binding of the APC-labeled gp41 MPER-tetramer >90%, confirming gp41 2F5 MPER binding to BALB/c B cells was indeed epitope-specific.

A determination was made as to whether this high frequency binding of BALB/c B cells was specific to the gp41 MPER region by incubating BALB/c splenocytes with several HIV-1 epitope-bearing B tetramers, including two gp120 V3 loop epitope-specific tetramers (B.con V3 and 62.19 V3), a gp41 immunodominant region-specific tetramer, and the gp41 2F5 MPER tetramer, or their respective scrambled counterparts (FIG. 9). Importantly, no tetramer other than gp41 2F5 MPER, including the gp41 immunodominant region-specific reagent, bound B cells at frequencies significantly higher than their respective scrambled controls. Also notable is that no significant binding above background was seen with a previously-described double DNA epitope-containing B cell tetramer (Newman et al, J. Immunol. Methods 272 (1-2):177-187 (2003)), a reagent that has been used to measure relative autoreactivities in B cell populations (Rice et al, Proc. Natl. Acad. Sci. USA 102(5):1608-1613 (2005)). Specifically, the lack of significant binding with this reagents suggests two things; first, the elevated binding by the gp41 2F5 MPER epitope to BALB/c cells cannot be attributed to the general increased autoreactivity reported to be associated with the BALB/c genetic background relative to C57BL/6 mice (Hardy et al, J. Exp. Med. 173(5):1213-1225 (1991)) and secondly, the background levels binding with the dsDNA tetramer reagent we observed were comparable to those previously reported in total naïve splenic populations (Rice et al, Proc. Natl. Acad. Sci. USA 102(5):1608-1613 (2005)), indicating that any potential variations in our staining conditions cannot sufficiently explain cross-reactive or “sticky” binding of BALB/c B cells to the gp41 2F5 MPER epitope.

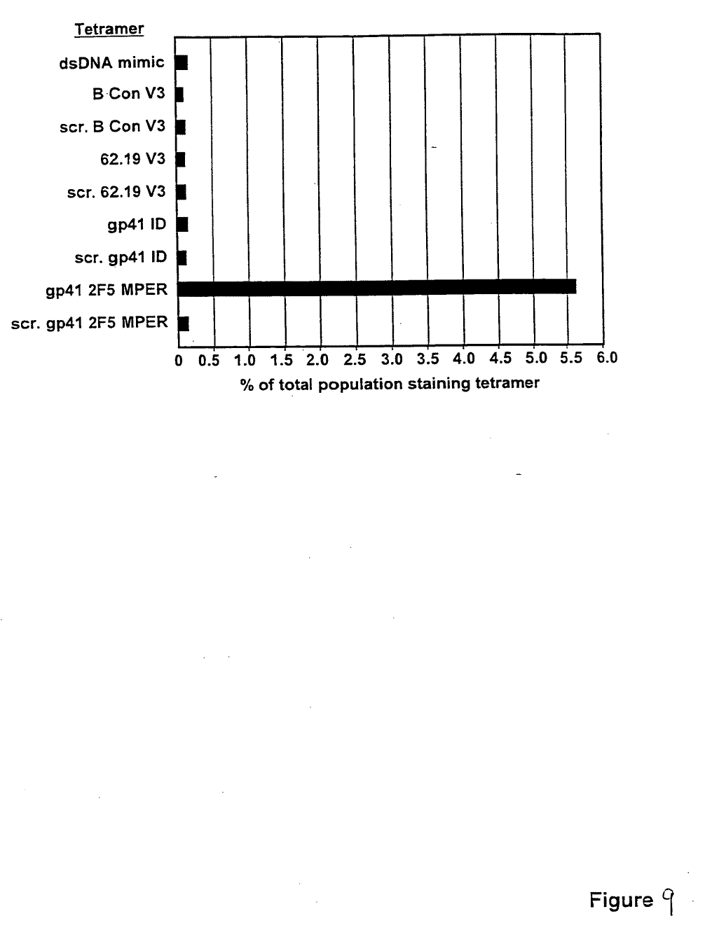

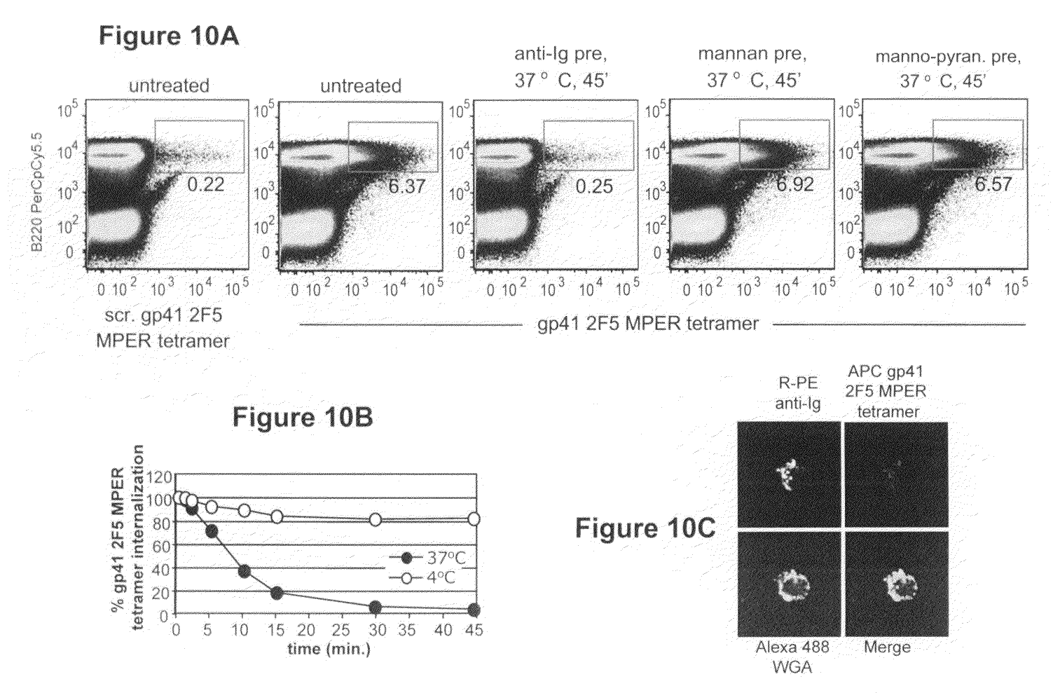

gp41 MPER 2F5 epitope-bearing tetramers interact with a significant subset of naïve BALB/c splenic B cells by specific binding through their B Cell Receptors (BCRs). Since the fraction of total naïve BALB/c B cells that bound gp41 MPER was much higher than would be anticipated for any given antigen-specific subpopulation, a series of experiments were performed to examine whether the gp41 2F5 MPER epitope specifically interacted with the B cell Receptor (BCR), or via other interactions with the B cell membrane, for example other surface receptors like, Mannose C-type Lectin-binding Receptors (MCLRs), which have been reported to interact with HIV-1 virions on B cells (He et al, J. Immunol. 176(7):3931-3941 (2006)). First, internalization experiments were conducted by flow cytometry (FIG. 10A). Indeed, rapid gp41 2F5 MPER tetramer downmodulation was seen following incubation with an anti-heavy chain+light chain constant region-specific Ig reagent for 30 minutes at 37° C. (FIG. 10A, middle panels). Significantly, comparable blocking/internalization of MCLRs was not seen (FIG. 10A, right panels). Tetramer downmodulation was rapid (>80% by 15 min), and was not observed upon incubation with anti-Ig reagent on ice suggesting that internalization via Fab regions, rather than blocking of sIg constant domains, accounted for diminished gp41 2F5 MPER binding (FIG. 10B). To further test if the gp41 2F5 MPER tetramer associated with BCR complexes, co-capping experiments were performed by immunofluorescence microscopy. Indeed, examples of gp41 2F5 MPER tetramer co-capping with BCRs within planes of BALB/c purified splenic B cells stimulated with both reagents at 37 degrees were observed (FIG. 10C). Taken together, the results demonstrate that the gp41 2F5 MPER epitope interacts with large fractions of BALB/c B cells largely, if not exclusively, though their BCRs, suggesting the gp41 2F5 MPER binds to BALB/c B cells as a B cell superantigen.

gp41 F5 MPER epitope interactions with the BCR of naïve mice map to the IgH^{a} locus, and likely occur in V_{H} framework regions. In addition to the greatly elevated fraction of B cells recognizing superantigens relative to conventional antigens, interactions between the BCR and B cell superantigens differ from those with conventional antigens in other several ways (reviewed in Silverman and Goodyear, Nat. Rev. Immunol. 6(6):465-475 (2006)). Most notable is the tendency of B cell superantigens to bind outside the antigen-binding pocket (comprised of V_{H} and V_{L} CDR3 region juxtapositions), to V framework regions along the sides of heavy chains, as well as the tendency to use restricted V_{H} families (in contrast to the full V_{H} repertoire utilized by B2 cells interacting with conventional antigens). The results demonstrating a significant fraction of BCR-gp41 2F5 MPER interactions present in naïve B cells, coupled with the results demonstrating internalization, but not blocking, of BCR-2F5 epitope interactions mediated by anti-Ig constant region-specific reagents, suggest that the 2F5 epitope is not binding to Fab V regions in a conventional antigen-specific manner, but in an allotype-restricted manner resembling B cell superantigen interactions with V_{H} framework regions.

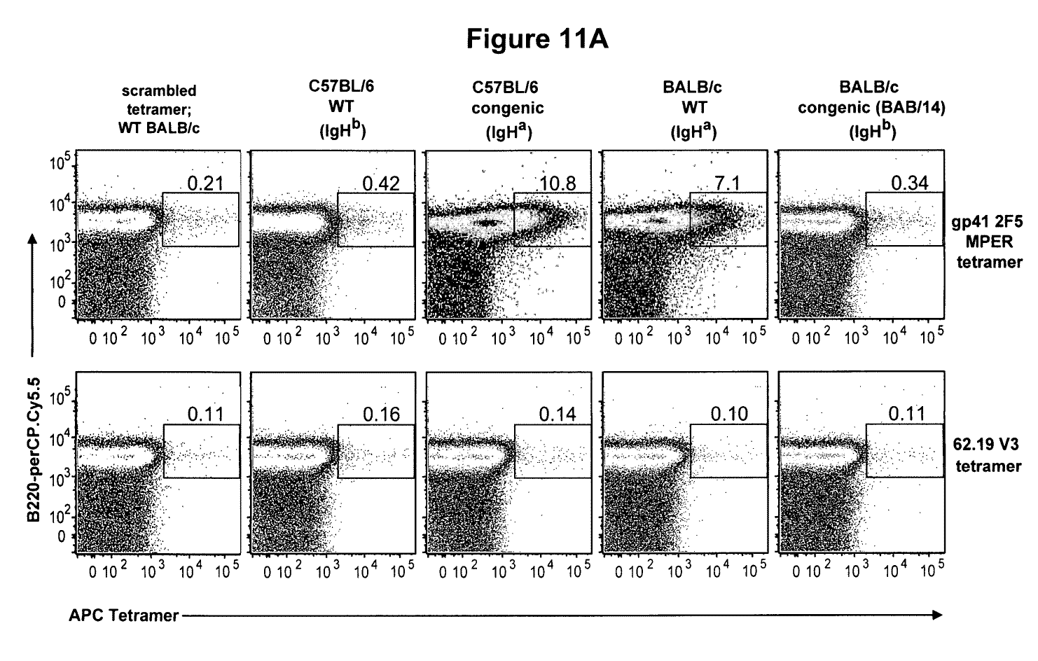

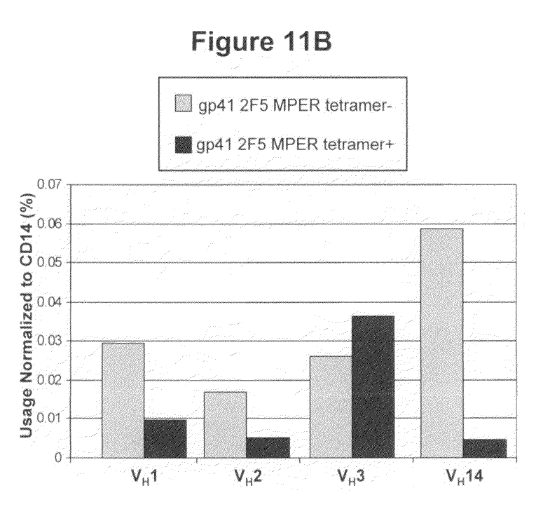

To test this possibility, two series of experiments were carried out. First, since one key difference between BALB/c and C57BL/6 strains is usage of IgH allotypic determinants, it was genetically tested whether the BCR-specific, strain-specific binding of naïve B splenocytes segregated with the BALB/c Ig heavy chain locus allotype (IgH^{a}). To do this, an examination was made of binding of total naïve splenic B cells from a series of congenic mouse strains, differing in allotypic determinants at their IgH locus, to the gp41 2F5 MPER epitope-specific tetramer (FIG. 11A, upper panels) and the control 62.19 V3 epitope-specific tetramer (FIG. 11A, lower panels). Indeed, it was found that the gp41 2F5 MPER epitope binding mapped to the IgH^{a} allotype, as the C57BL/6 congenic strain bearing the IgH^{a} allotype restores gp41 2F5 MPER binding (FIG. 11A, upper middle panel), while conversely, the BALB/c congenic strain bearing the IgH^{b} allotype completely loses gp41 2F5 MPER binding (FIG. 11A, upper right panel).

Secondly, a comparison was made of usage of V_{H} families in gp41 2F5 MPER tetramer-reactive (+) and gp41 2F5 MPER tetramer non-reactive (−) bulk-sorted total splenic B cell populations from naïve BALB/c mice using quantitative PCR to sample four V_{H} families spanning proximal (V_{H}2, V_{H}14), intermediate (V_{H}3), and distal (V_{H}1) portions of the heavy chain V cluster (FIG. 1 lB). Importantly, relative to gp41 2F5 MPER tetramer non-binding B cell fractions, significantly diminished binding was found in gp41 2F5 MPER tetramer-binding B cell fractions in three of four families assessed. This includes the very large J558 (V_{H}1) family, which is preferentially utilized in IgH^{a allotype-specific mouse strains (Sheehan and Brodeur, EMBO J.} 8(8):2313-2320 (1989)). The observed skewing in V_{H}1, V_{H}2, and V_{H}14 families not only suggests that the gp41 2F5 MPER interacts with V_{H} framework regions of the BCR Fab regions, but also implies this interaction involves preferential usage of either a single specific V_{H} family or a restricted set of V_{H} families.

Taken together, the above results are consistent with the binding of the gp41 2F5 MPER epitope to naïve splenic B cells as an IgH^{a}-restricted V_{H} framework-specific B cell superantigen.

Distinct residues within the gp41 2F5 MPER epitope are required for B cell superantigen interactions from those required for 2F5 Mab binding and neutralization. To determine what residues in the gp41 2F5 MPER are involved in superantigen binding to BALB/c (IgH^{a}) B cells, a series of tetramer mutants made from peptides with alanine substitutions in sequential sites along the gp41 2F5 MPER epitope were created (FIG. 12A). To compare binding of the MPER as a B cell superantigen with that to the Mab 2F5, these tetramers were then used to map binding of Mabs 2F5, 13H11, and 5A9 by surface plasmon resonance analysis, and binding to naïve splenic BALB/c B cells by flow cytometry (FIG. 12B). Consistent with previous studies (Zwick et al, J. Virol. 79(2):1252-1261 (2005)), it was found that binding of the 2F5 Mab occurs in the neutralization-sensitive residues D and W within the core 2F5 epitope ELDKWA (FIG. 12B, top panel). In contrast, B cell superantigen binding activity maps to residues distinct from these 2F5 neutralization-sensitive sites, mapping instead to an N-terminal Q residue, and three consecutive C-terminal residues (L, W, and N), both located outside the core 2F5 epitope (FIG. 12B, middle panel). It is, therefore, proposed that two distinct interactions occur between the gp41 2F5 MPER epitope and Ig: superantigen binding interactions with surface Ig VH regions of naïve B cells and antigen-specific binding interactions with CDR3 regions of MPER neutralizing antibodies such as 2F5.

Interestingly, two of the C-terminal residues (L and W) required for B cell superantigen-binding activity are also required for binding to two non-neutralizing Abs, 13H11 and 5A9, (derived from BALB/c mice immunized with Env), while others are unique to either MPER-BCR superantigen or MPER-13H11/5A9 interactions (compare FIG. 12B, middle panel with lower panel). Further studies will determine the relationship of the B cell superantigen MPER interactions and non-neutralizing MPER Abs. Since the residues for both phenomena are overlapping, but also unique, this suggests that if superantigen interactions are involved in eliciting non-neutralizing antibody responses, they would be necessary, but not sufficient (i.e., other immune response influences would also be required). In this context, further studies, including immunization studies in congenic strains, will determine the relative requirement of the IgH^{a} locus and other strain-specific differences in eliciting BALB/c-specific non-neutralizing Ab responses.

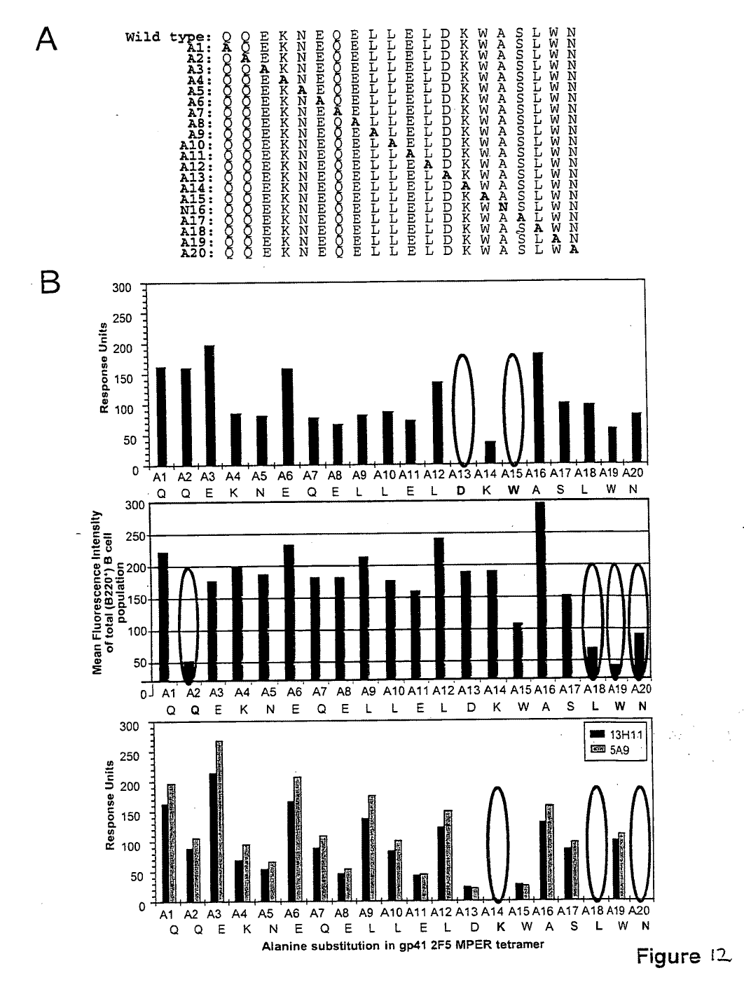

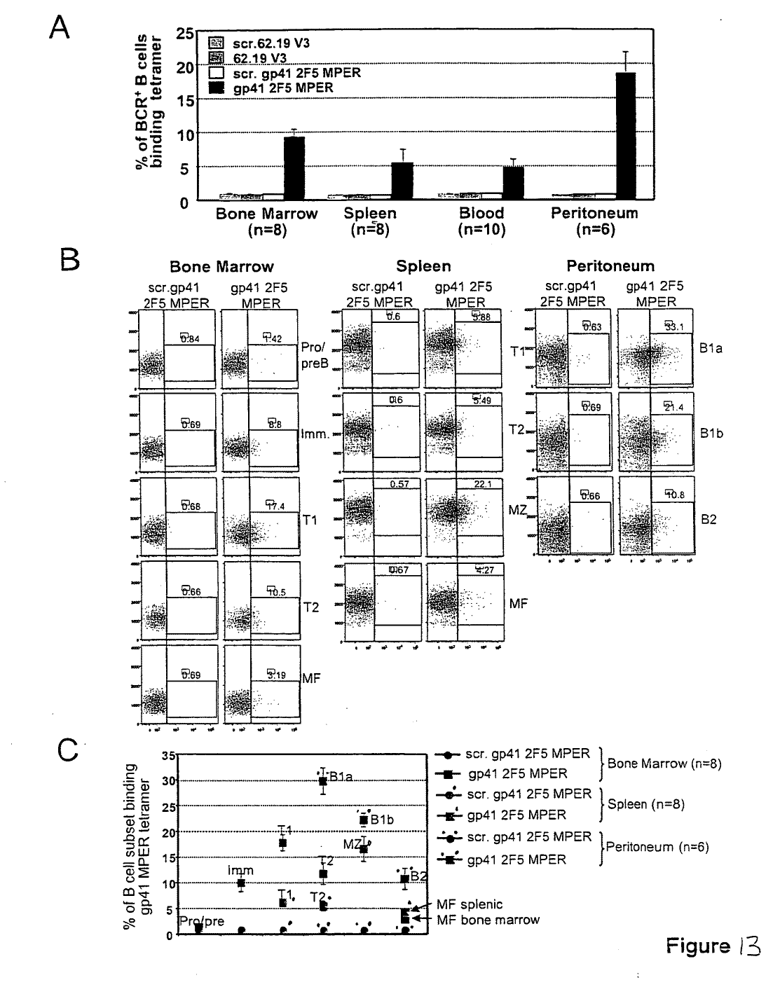

Differential gp41 2F5 MPER epitope reactivities within and between BALB/c and C57BL/6 B cell subsets suggest that MPER-BCR interactions are controlled both by superantigen binding and by developmentally-regulated B cell tolerance mechanisms. To determine if IgH^{a} allotype-restricted superantigen interactions of the gp41 2F5 MPER epitope with BCRs from naïve BALB/c splenocytes was uniformly distributed across primary and secondary lymphoid tissue subsets or, alternatively, preferentially segregated to certain tissues and/or within particular B cell subsets within the total naïve B cell repertoire, the frequencies of gp41 2F5 MPER tetramer-reactive B cells within total bone marrow, splenic, peritoneal, and blood total B cell populations (FIG. 13A) and within specific B cell subsets were established (FIGS. 13B, C, and Tables 9 and 10). To establish frequencies of gp41 2F5 MPER-reactive B cell within each subset, a measurement was made of reactivity to the gp41 2F5 MPER epitope in two independent ways: either as the percentage of APC-labeled gp41 MPER tetramer-reactive cells (FIG. 13A-C and Table 10) or as APC mean fluorescence intensities of entire populations (Table 9).

TABLE 9
Relative reactivity of B cell subsets from naï
ve BALB/c mice to the 2F5 epitope as calculated by MFIs of
populations stained with gp41 2F5 MPER tetramer^{a}

| | | APC Mean Fluorescence Intensity ± SD | |
|---|---|---|---|
| | | tetramer | |
| | | scr.gp41 | gp41 2F5 |
| Tissue | B cell subset^{b} | 2F5 MPER | MPER |
| Bone marrow | Pro/pre | 59.2 ± 5.2 | 64.2 ± 8.2 |
| | Immature | 54.8 ± 4.6 | 105.1 ± 11.5 |
| | T1 | 61.4 ± 5.8 | 219.4 ± 21.1^{c} |
| | T2 | 62.5 ± 6.6 | 130.5 ± 17.6 |
| | MF | 65.2 ± 4.1 | 77.4 ± 8.1 |
| Spleen | T1 | 24.5 ± 3.2 | 44.1 ± 6.8 |
| | T2 | 23.6 ± 2.8 | 40.7 ± 6.2 |
| | MZ | 27.2 ± 3.8 | 160.8 ± 13.8 |
| | B1a | 23.6 ± 4.6 | 25.1 ± 3.6 |
| | MF | 22.8 ± 3.1 | 32.6 ± 4.1 |
| Peritoneum | B1a | 66.3 ± 6.2 | 385.2 ± 25.2 |
| | B1b | 68.1 ± 4.9 | 232.9 ± 22.9 |
| | B2 | 60.7 ± 5.1 | 145.2 ± 24.6 |

^{a}MFI values (+SD) are from a representative experiment with 5 mice. Similar trends across and within tissues/subsets were seen in several independent experiments, which were calculated separately due to variation in baseline MFIs between stains.
^{b}B cell subsets from total B cell populations (singlet, live, lin−, CD19⁺ and/or B220⁺ gated cells) were defined as: bone marrow Progenitor/precursor (Pro/pre; B220^{lo}IgM⁻IgD⁻CD21⁻CD23⁻), bone marrow Immature (B220^{lo}IgM^{lo}IgD⁻CD21⁻CD23⁻), bone marrow/splenic transitional 1 (T1; B220^{int}IgM^{hi}IgD⁻CD21^{+/-}CD23^{+/-}), bone marrow/splenic transitional 2 (T2; B220^{hi}IgM^{int/hi}IgD^{lo}CD21⁺CD23⁺), bone marrow/splenic B2 mature follicular (MF; B220^{hi}IgM^{int}IgD^{hi}CD21⁺⁺CD23⁺⁺), splenic marginal zone (MZ; B220^{int}IgM^{hi}IgD^{lo}CD21⁺⁺⁺CD23⁻), splenic B1a (CD5⁺), peritoneal B1a (B220^{lo}CD5⁺CD1d^{hi}IgM^{hi}), peritoneal B1b (B220^{lo}CD5⁻CD1d^{hi}IgM^{hi}), and peritoneal B2 (B220^{hi}CD5⁻CD1d^{lo}IgM^{lo}).
^{c}Bolded items represent significant (p < 0.05) increases in gp41 2F5 MPER-binding subsets relative to corresponding scr. gp41 MPER-binding subsets and to total (BCR⁺ B220⁺) gp41 2F5 MPER-binding B cell populations from corresponding tissues.

TABLE 10
Relative reactivity of naive BALB/c and C57BL/6 B cell subsets to the 2F5
epitope as calculated by % of gp41 2F5 MPER tetramer-binding events^{a}

| | | % of subset binding tetramer ± SEM | | | |
|---|---|---|---|---|---|
| | | BALB/c^{b} | | C57BL/6^{c} | |
| | | tetramer | | tetramer | |
| | B cell | scr.gp41 | gp41 2F5 | scr.gp41 | gp41 2F5 |
| Tissue | subset | 2F5 MPER | MPER | 2F5 MPER | MPER |
| Bone marrow | Pro/pre | 0.28 ± 0.02 | 1.34 ± 0.15 | 0.06 ± 0.01 | 0.05 ± 0.01 |
| | Immature | 0.23 ± 0.01 | 9.95 ± 1.77 | 0.10 ± 0.02 | 0.30 ± 0.04 |
| | T1 | 0.20 ± 0.03 | 17.73 ± 1.53^{d} | 0.12 ± 0.02 | 0.62 ± 0.10^{d} |
| | T2 | 0.22 ± 0.02 | 11.69 ± 2.11^{d} | 0.11 ± 0.01 | 0.54 ± 0.09^{d} |
| | MF | 0.24 ± 0.04 | 2.81 ± 0.75 | 0.09 ± 0.02 | 0.15 ± 0.06 |
| Spleen | T1 | 0.14 ± 0.04 | 6.09 ± 0.70 | 0.08 ± 0.01 | 0.24 ± 0.06 |
| | T2 | 0.11 ± 0.03 | 5.32 ± 0.88 | 0.10 ± 0.02 | 0.21 ± 0.07 |
| | MZ | 0.13 ± 0.05 | 16.46 ± 2.41^{e} | 0.09 ± 0.01 | 0.25 ± 0.04 |
| | MF | 0.17 ± 0.02 | 4.16 ± 0.48 | 0.11 ± 0.02 | 0.16 ± 0.02 |
| Peritoneum | B1a | 0.34 ± 0.05 | 29.70 ± 258^{e} | 0.14 ± 0.03 | 0.52 ± 0.08 |
| | B1b | 0.33 ± 0.06 | 22.11 ± 1.32 | 0.13 ± 0.02 | 0.50 ± 0.13 |
| | B2 | 0.39 ± 0.04 | 10.65 ± 2.03 | 0.15 ± 0.03 | 0.32 ± 0.09 |

^{a}Reactivity values (% ± SEM) were determined as the percentage of each subset positive for staining gp41 2F5 MPER tetramers within “gp41 MPER gates”, defined independently in each tissue by setting the baseline to include all events greater than those observed in FMO controls (i.e. staining without tetramer).
^{b}Values (+SD) are combined from three independent experiments with 5 mice each.
^{c}Values (+SD) is from one experiment with 5 mice, done in conjunction with staining of BALB/c mice.
^{d}Items bolded in blue represent significant (p < 0.05) increases of gp41 2F5 MPER-binding subsets seen in BALB/c and C57BL/6 strains compared to total B cell populations. Increases were deemed significant only if differences were seen both relative to corresponding scr. gp41 2F5 MPER-binding subsets and to total (BCR⁺ B220⁺) gp41 2F5 MPER-binding B cell populations from corresponding tissues.
^{e}Items bolded in red represent significant (p < 0.05) increases of gp41 2F5 MPER-binding subsets compared to total B cell populations unique to BALB/c mice.

Using either measurement approach, gp41 2F5 MPER epitope reactivity was found to preferentially map to IgH^{a} B cell subsets in a tissue-specific and developmentally-regulated manner. In particular, innate and developmentally immature “polyreactive” B cell subsets exhibited significantly elevated frequencies of 2F5 epitope reactive populations (for example, see FIG. 13C and Table 10: ˜11-18%, ˜16%, ˜22%, and ˜30% in bone marrow transitional, splenic marginal zone, and peritoneal B1a and B1b subsets, respectively). In contrast, mature follicular B2 subsets (splenic and bone marrow recirculating) as well as splenic transitional B cell subsets exhibited diminished binding (˜3-4% and 5-6%, respectively).

To examine if gp41 2F5 MPER epitope reactivity across B cell subsets mapped similarly on the IgH^{b} allotype, reactivity to gp41 2F5 MPER tetramers was measured by measuring the percentage of APC-labeled gp41 2F5 MPER tetramer-reactive cells in C57BL/6 bone marrow, splenic, and peritoneal B cell subsets (Table 10). Interestingly, while significant increases in bone marrow transitional 2F5 epitope-reactive population frequencies were also observed in C57BL/6 mice (albeit <1 order of magnitude proportionally lower than observed in BALB/c mice; Table 10, “d” highlighted items), significant increases were not observed in C57BL/6 innate mature B cell subsets, i.e., splenic marginal zone (and to a lesser extent, peritoneal B1a B cells), unlike those significant increases seen in corresponding BALB/c subsets (Table 10, “e” highlighted items). Another potential difference in 2F5 reactivity observed between the two strains was qualitative in nature, namely in binding intensity. In particular, 2F5 reactivity of BALB/c subsets appears to shift the entire population as a shoulder (i.e. as a tightly-scattered binding continuum; see FIG. 13B and Table 9), whereas binding in C57BL/6 appeared to show reactivity of a small subset scattering much further out in the APC channel (data not shown). Further genetic studies involving sequencing of individual will determine if these binding differences reflect differential interaction of the 2F5 epitope with the BCR in these strains, for example, the occurrence of framework region-specific versus antigen recognition pocket-specific binding in BALB/c and C57BL/6 binding, respectively.

It is proposed that the differential binding patterns to the gp41 2F5 MPER epitope in the B cell subsets of BALB/c and C57BL/6 strains reveals an inherent susceptibility of the endogenous 2F5-reactive mouse B cell repertoire to at least two distinct tolerance mechanisms: i) an allotype-independent, developmentally-regulated tolerizing mechanism in the bone marrow (or in the T1/T2 splenic compartment), reflected by the elevated bone marrow T1/T2 cell binding in both BALB/c and C57BL/6 strains, ii) an IgH^{b allotype-specific tolerizing mechanism inherent to all subsets, for example an endogenous IgH}^{b} specific superantigen (that cross-reacts with the 2F5 MPER epitpope), reflected by the uniformly higher superantigen-mediated binding across all IgH^{a} subsets, and possibly, iii) the same or a different IgH^{b}-specific endogenous superantigen selectively targeting innate subsets, reflected by further preferential elevations in IgH^{a} splenic marginal zone subsets.

### Example 3

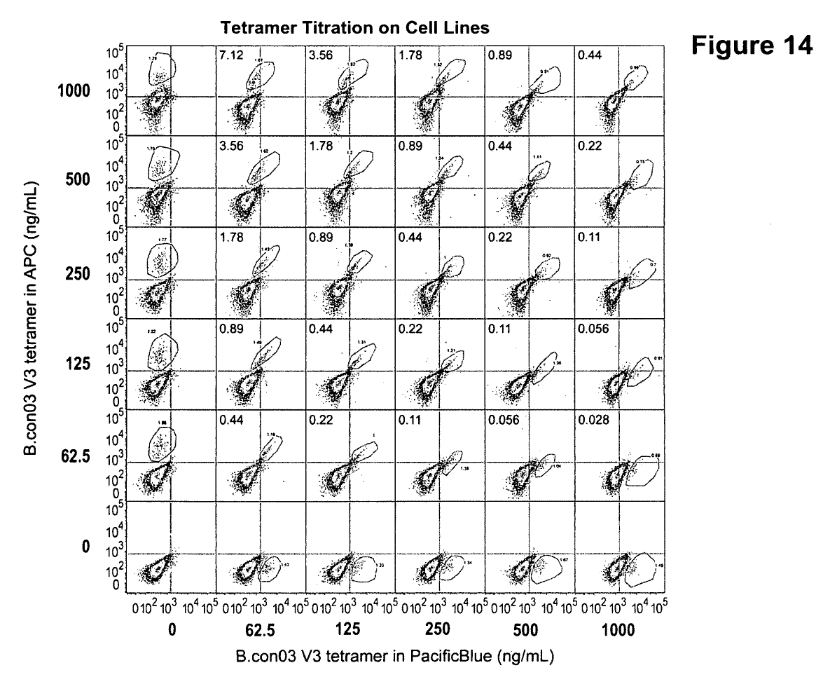

A cell mixture made of approximately 1% anti-V3 loop mAb F39F and 99% murine myeloma cell line P3X63/Ag8 was stained using serial dilutions of B.con03 V3 tetramer labeled with allophycocyanin (APC) or PacificBlue singly and in combination. In FIG. 14, the axes list the staining concentrations of each tetramer in ng/mL. The numbers in the upper left corner of the bivariate plots show the molar ratio of the tetramers for a given combination. Doubly stained F39F cells are found along a diagonal in the plots, with the position of the line shifting relative to the concentration of the tetramers. Optimal separation for this experiment is found at 125 ng/mL of APC tetramer and 62.5 ng/mL of PacificBlue tetramer. Increasing concentration of tetramer did not increase the brightness of the positive signal (indicating saturation) but did increase the fluorescence of the negative population, ultimately obscuring the positive population at the highest concentration of tetramers.

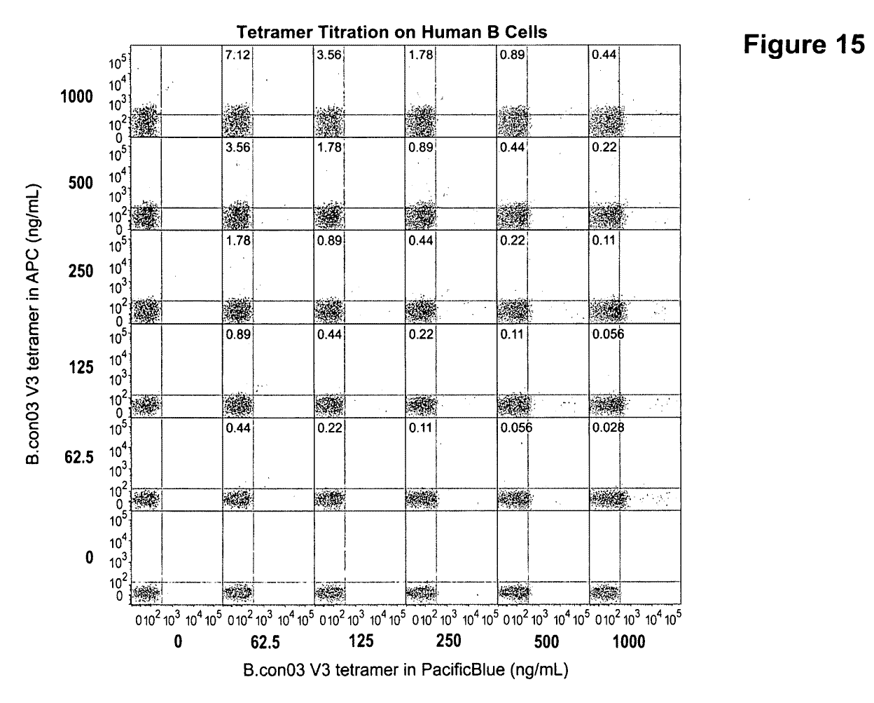

Human PBMC from a chronically HIV-1 infected subject were obtained by leukopheresis under an IRB-approved protocol. Samples were stained using the same protocol as for FIG. 14. The plots shown in FIG. 15 are gated on CD19+ cells (B cells). Similar to the result for FIG. 14, increasing the concentration of tetramer does not increase the positive signal but does cause a shift of the negative population that begins to obscure the double positive population. (See FIG. 15.)

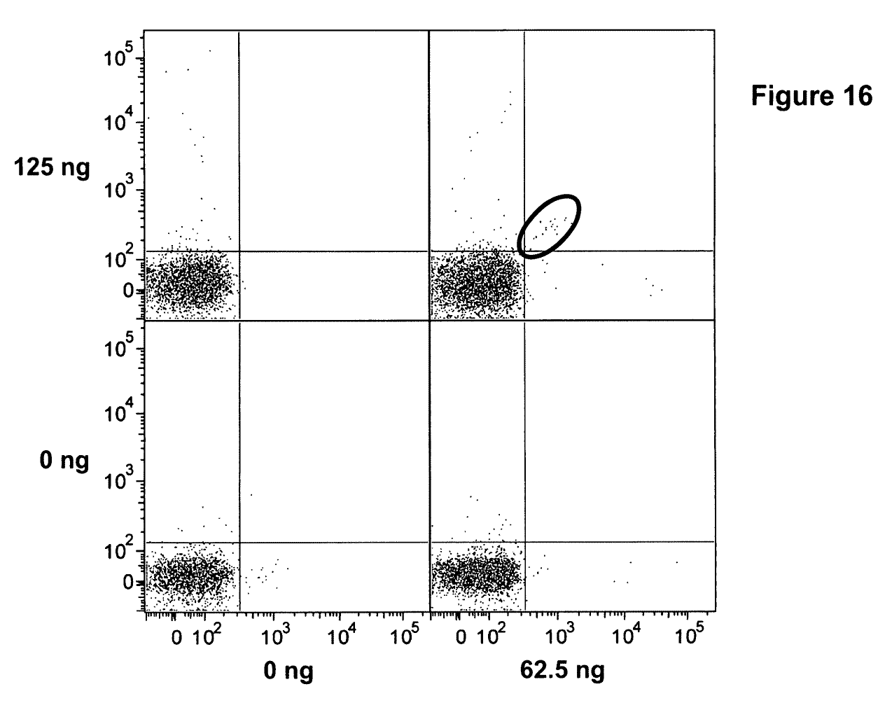

Shown in FIG. 16 are four plots from FIG. 15, corresponding to combinations of 0 or 1 25 ng/mL of APC-labeled tetramer and 0 or 62.5 ng/mL of PacificBlue-labeled tetramer. In the upper right panel the red oval outlines the dual tetramer labeled cells.

All documents and other information sources cited above are hereby incorporated in their entirety by reference.

What is claimed is:

1. A method of isolating epitope-specific B cells, wherein said epitope is a broadly neutralizing HIV-1 epitope, comprising:
i) contacting a sample comprising said epitope-specific B cells with: a) a B cell tetramer comprising said epitope complexed with a binding pair, b) said binding pair free of said epitope, and c) a tetramer comprising a random amino acid sequence complexed with said binding pair, wherein said contacting is effected under conditions such that said epitope-specific B-cells can bind to said B cell tetramer, and
ii) separating said epitope-specific B-cells bound to said B cell tetramer from said mixture resulting from step (i) so that said isolation is effected.

2. The method according to claim 1 wherein said sample is a biological sample obtained from an HIV-1 infected individual or from an individual immunized against HIV-1.

3. The method according to claim 1 wherein said B cells are CD19⁺ B cells

4. The method according to claim 1 wherein said epitope is an epitope of the membrane proximal external region (MPER) of gp120 recognized by human monoclonal antibody 2F5, 4E10 or Z13.

5. The method according to claim 1 wherein said epitope comprises the sequence TRPNNNTRKSIHIGPGRAFYTTGEIIGDIRQAH (SEQ ID NO: 88), TRPNNNTRKSIHIGPGRAFYATE (SEQ ID NO: 89), KQLQARVLAVERYLKDQQLLGIWGCSGKLICTTAV (SEQ ID NO: 90) or QQEKNEQELLELDKWASLWN (SEQ ID NO: 59).

6. The method according to claim 1 wherein said epitope is a non-naturally occurring epitope.

7. The method according to claim 6 wherein said epitope comprises the sequence of QQEKNEQELLELDAWASLWN (SEQ ID NO: 73), QQEKNEQELLELDKWASAWN (SEQ ID NO: 2), and QQEKNEQELLELDKWASLWA (SEQ ID NO: 70).

8. The method according to claim 1 wherein at least one member of said binding pair bears a detectable label.

9. The method according to claim 8 wherein said detectable label is a fluorochrome.

10. The method according to claim 9 wherein said fluorochrome is allophycocyanin or PacificBlue.

11. The method according to claim 1 wherein said binding pair comprises biotin and streptavidin.

12. The method according to claim 11 wherein steptavidin bears a detectable label.

13. The method according to claim 1 wherein the amino acid composition of said random amino acid sequence is the same as the amino acid composition of said epitope, the order of said amino acids in said random amino acid sequence being scrambled with respect to the order of said amino acids in said epitope.

14. The method according to claim 1 wherein said epitope is covalently bound to a member of said binding pair via a spacer molecule.

15. The method according to claim 14 wherein said spacer molecule comprises 3-5 G's or —(CH₂)₅—.

16. The method according to claim 1 wherein, in step (ii), said separation is effected using flow cytometry.

17. The method according to claim 1 further comprising isolating immunoglobulin encoding sequences from said isolated epitope-specific B-cells resulting from step (ii).

18. The method according to claim 17 wherein said encoding sequences are isolated by reverse transcription and polymerase chain reaction.

19. A method of monitoring the efficacy of an anti-HIV-1 immunization protocol comprising:
i) administering to a subject an antigen capable of inducing an anti-HIV-1 immune response,
ii) obtaining a B-cell-containing biological sample from said subject,
ii) contacting said sample with:
a) a B cell tetramer comprising a broadly neutralizing HIV-1 epitope complexed with a binding pair,
b) said binding pair free of said epitope, and
c) a tetramer comprising a random amino acid sequence complexed with said binding pair,
wherein said contacting is effected under conditions such that B-cells specific for said epitope present in said sample can bind to said B cell tetramer, and
iii) isolating said epitope-specific B-cells bound to said B cell tetramer from said mixture resulting from step (ii) and thereby determining the presence of said epitope-specific B-cells bound to said B cell tetramer,
wherein the presence of said epitope-specific B-cells bound to said B cell tetramer indicates said protocol is efficacious.

20. A tetramer comprising a peptide comprising an amino acid sequence selected from the group consisting of QQEKNEQELLELDAWASLWN (SEQ ID NO: 73), QQEKNEQELLELDKWASAWN (SEQ ID NO: 2) and QQEKNEQELLELDKWASLWA (SEQ ID NO: 78), bound to a member of a binding pair.

21. A composition comprising the tetramer according to claim 20 and a carrier.


SEQUENCE LISTING (90 sequences)

SEQ ID NO: 1 - protein, 6 aa, Human immunodeficiency virus
ELDKWA

SEQ ID NO: 2 - protein, 20 aa, Artificial Sequence
  Description of Artificial Sequence Synthetic peptide
QQEKNEQELLELDKWASAWN

SEQ ID NO: 3 - protein, 20 aa, Artificial Sequence
  Description of Artificial Sequence Synthetic peptide
QQEKNEQELLELDKWASAAN

SEQ ID NO: 4 - protein, 20 aa, Artificial Sequence
  Description of Artificial Sequence Synthetic peptide
QAEKNEQELLELDKAASLWN

SEQ ID NO: 5 - protein, 20 aa, Artificial Sequence
  Description of Artificial Sequence Synthetic peptide
QQEKNEQELLELDKAASLAN

SEQ ID NO: 6 - protein, 20 aa, Artificial Sequence
  Description of Artificial Sequence Synthetic peptide
QQEKNEQELLELAAWASLWN

SEQ ID NO: 7 - protein, 20 aa, Artificial Sequence
  Description of Artificial Sequence Synthetic peptide
QQEKNEQELLELAKWASLWA

SEQ ID NO: 8 - protein, 20 aa, Artificial Sequence
  Description of Artificial Sequence Synthetic peptide
QQEKNEQELLELDKAASAWN

SEQ ID NO: 9 - protein, 22 aa, Artificial Sequence
  Description of Artificial Sequence Synthetic peptide
QQEKNEQELLELDKWASAANWF

SEQ ID NO: 10 - protein, 36 aa, Artificial Sequence
  Description of Artificial Sequence Synthetic peptide
GGGTRPNNNTRKSIHIGPGRAFYTTGEIIGDIRQAH

SEQ ID NO: 11 - protein, 36 aa, Artificial Sequence
  Description of Artificial Sequence Synthetic peptide
GGGITIDNIGHHNRITFRAANTRPISGQRGEYPGKT

SEQ ID NO: 12 - protein, 26 aa, Artificial Sequence
  Description of Artificial Sequence Synthetic peptide
GGGTRPNNNTRKSIHIGPGRAFYATE

SEQ ID NO: 13 - protein, 26 aa, Artificial Sequence
  Description of Artificial Sequence Synthetic peptide
GGGNPATISRTIGHNFRYTPAREKNG

SEQ ID NO: 14 - protein, 38 aa, Artificial Sequence
  Description of Artificial Sequence Synthetic peptide
GGGKQLQARVLAVERYLKDQQLLGIWGCSGKLICTTAV

SEQ ID NO: 15 - protein, 38 aa, Artificial Sequence
  Description of Artificial Sequence Synthetic peptide
GGGQLDSIQKEVYQLGRQLVWCTARLATKLVGKACGIL

SEQ ID NO: 16 - protein, 23 aa, Artificial Sequence
  Description of Artificial Sequence Synthetic peptide
GGGQQEKNEQELLELDKWASLWN

SEQ ID NO: 17 - protein, 23 aa, Artificial Sequence
  Description of Artificial Sequence Synthetic peptide
GGGNKEQDQAEESLQLWEKLNWL

SEQ ID NO: 18 - protein, 15 aa, Artificial Sequence
  Description of Artificial Sequence Synthetic peptide
GGGGGDWEYSVWLSN

SEQ ID NO: 19 - DNA, 20 bp, Artificial Sequence
  Description of Artificial Sequence Synthetic primer
atggagtcgggaaggaagtc

SEQ ID NO: 20 - DNA, 19 bp, Artificial Sequence
  Description of Artificial Sequence Synthetic primer
tcacggacgttgggtggta

SEQ ID NO: 21 - DNA, 19 bp, Artificial Sequence
  Description of Artificial Sequence Synthetic primer
tcacggaggtggcattgga

SEQ ID NO: 22 - DNA, 19 bp, Artificial Sequence
  Description of Artificial Sequence Synthetic primer
caggcgatgaccacgttcc

SEQ ID NO: 23 - DNA, 19 bp, Artificial Sequence
  Description of Artificial Sequence Synthetic primer
catgcgacgaccacgttcc

SEQ ID NO: 24 - DNA, 20 bp, Artificial Sequence
  Description of Artificial Sequence Synthetic primer
aggtgtgcacgccgctggtc

SEQ ID NO: 25 - DNA, 20 bp, Artificial Sequence
  Description of Artificial Sequence Synthetic primer
gcaggcacacaacagaggca

SEQ ID NO: 26 - DNA, 17 bp, Artificial Sequence
  Description of Artificial Sequence Synthetic primer
aggccactgtcacagct

SEQ ID NO: 27 - DNA, 20 bp, Artificial Sequence
  Description of Artificial Sequence Synthetic primer
ccatggactggacctggagg

SEQ ID NO: 28 - DNA, 19 bp, Artificial Sequence
  Description of Artificial Sequence Synthetic primer
atggacatactttgttcca

SEQ ID NO: 29 - DNA, 20 bp, Artificial Sequence
  Description of Artificial Sequence Synthetic primer
ccatggagtttgggctgagc

SEQ ID NO: 30 - DNA, 20 bp, Artificial Sequence
  Description of Artificial Sequence Synthetic primer
atgaaacacctgtggttctt

SEQ ID NO: 31 - DNA, 20 bp, Artificial Sequence
  Description of Artificial Sequence Synthetic primer
atggggtcaaccgccatcct

SEQ ID NO: 32 - DNA, 20 bp, Artificial Sequence
  Description of Artificial Sequence Synthetic primer
atgtctgtctccttcctcat

SEQ ID NO: 33 - DNA, 17 bp, Artificial Sequence
  Description of Artificial Sequence Synthetic primer
gctcagctcctggggct

SEQ ID NO: 34 - DNA, 17 bp, Artificial Sequence
  Description of Artificial Sequence Synthetic primer
ggaarccccagcdcagc

SEQ ID NO: 35 - DNA, 19 bp, Artificial Sequence
  Description of Artificial Sequence Synthetic primer
ctsttsctytggatctctg

SEQ ID NO: 36 - DNA, 17 bp, Artificial Sequence
  Description of Artificial Sequence Synthetic primer
ctsctgctctgggytcc

SEQ ID NO: 37 - DNA, 20 bp, Artificial Sequence
  Description of Artificial Sequence Synthetic primer
cgaygaccacgttcccatct

SEQ ID NO: 38 - DNA, 24 bp, Artificial Sequence
  Description of Artificial Sequence Synthetic primer
ctgttatcctttgggtgtctgcac

SEQ ID NO: 39 - DNA, 20 bp, Artificial Sequence
  Description of Artificial Sequence Synthetic primer
cgcctgagttccacgacacc

SEQ ID NO: 40 - DNA, 20 bp, Artificial Sequence
  Description of Artificial Sequence Synthetic primer
ccgacggggaattctcacag

SEQ ID NO: 41 - DNA, 20 bp, Artificial Sequence
  Description of Artificial Sequence Synthetic primer
gaggcagttccagatttcaa

SEQ ID NO: 42 - DNA, 17 bp, Artificial Sequence
  Description of Artificial Sequence Synthetic primer
cctgggcccagtctgtg

SEQ ID NO: 43 - DNA, 18 bp, Artificial Sequence
  Description of Artificial Sequence Synthetic primer
ctcctcasyctcctcact

SEQ ID NO: 44 - DNA, 18 bp, Artificial Sequence
  Description of Artificial Sequence Synthetic primer
ggcctcctatgwgctgac

SEQ ID NO: 45 - DNA, 23 bp, Artificial Sequence
  Description of Artificial Sequence Synthetic primer
gttctgtggtttcttctgagctg

SEQ ID NO: 46 - DNA, 18 bp, Artificial Sequence
  Description of Artificial Sequence Synthetic primer
acagggtctctctcccag

SEQ ID NO: 47 - DNA, 19 bp, Artificial Sequence
  Description of Artificial Sequence Synthetic primer
acaggtctctgtgctctgc

SEQ ID NO: 48 - DNA, 17 bp, Artificial Sequence
  Description of Artificial Sequence Synthetic primer
ccctctcscagsctgtg

SEQ ID NO: 49 - DNA, 19 bp, Artificial Sequence
  Description of Artificial Sequence Synthetic primer
tcttgggccaattttatgc

SEQ ID NO: 50 - DNA, 19 bp, Artificial Sequence
  Description of Artificial Sequence Synthetic primer
attcycagrctgtggtgac

SEQ ID NO: 51 - DNA, 17 bp, Artificial Sequence
  Description of Artificial Sequence Synthetic primer
cagtggtccaggcaggg

SEQ ID NO: 52 - protein, 21 aa, Artificial Sequence
  Description of Artificial Sequence Synthetic peptide
METDTLLLWVLLLWVPGSTGD

SEQ ID NO: 53 - DNA, 31 bp, Artificial Sequence
  Description of Artificial Sequence Synthetic primer
agtaatcaattacggggtcattagttcatag

SEQ ID NO: 54 - DNA, 32 bp, Artificial Sequence
  Description of Artificial Sequence Synthetic primer
tccccagcatgcctgctattgtcttcccaatc

SEQ ID NO: 55 - protein, 22 aa, Human immunodeficiency virus
RPNNNTRKSIHIGPGRAFYATE

SEQ ID NO: 56 - protein, 22 aa, Human immunodeficiency virus
RPSNNTRKGIHLGPGRAIYATE

SEQ ID NO: 57 - protein, 22 aa, Human immunodeficiency virus
RPNNNTRKSIQIGPGRAFYTTG

SEQ ID NO: 58 - protein, 22 aa, Human immunodeficiency virus
RPNNNTRKSINIGPGRAFYTTG

SEQ ID NO: 59 - protein, 20 aa, Human immunodeficiency virus
QQEKNEQELLELDKWASLWN

SEQ ID NO: 60 - protein, 20 aa, Artificial Sequence
  Description of Artificial Sequence Synthetic peptide
AQEKNEQELLELDKWASLWN

SEQ ID NO: 61 - protein, 20 aa, Artificial Sequence
  Description of Artificial Sequence Synthetic peptide
QAEKNEQELLELDKWASLWN

SEQ ID NO: 62 - protein, 20 aa, Artificial Sequence
  Description of Artificial Sequence Synthetic peptide
QQAKNEQELLELDKWASLWN

SEQ ID NO: 63 - protein, 20 aa, Artificial Sequence
  Description of Artificial Sequence Synthetic peptide
QQEANEQELLELDKWASLWN

SEQ ID NO: 64 - protein, 20 aa, Artificial Sequence
  Description of Artificial Sequence Synthetic peptide
QQEKAEQELLELDKWASLWN

SEQ ID NO: 65 - protein, 20 aa, Artificial Sequence
  Description of Artificial Sequence Synthetic peptide
QQEKNAQELLELDKWASLWN

SEQ ID NO: 66 - protein, 20 aa, Artificial Sequence
  Description of Artificial Sequence Synthetic peptide
QQEKNEAELLELDKWASLWN

SEQ ID NO: 67 - protein, 20 aa, Artificial Sequence
  Description of Artificial Sequence Synthetic peptide
QQEKNEQALLELDKWASLWN

SEQ ID NO: 68 - protein, 20 aa, Artificial Sequence
  Description of Artificial Sequence Synthetic peptide
QQEKNEQEALELDKWASLWN

SEQ ID NO: 69 - protein, 20 aa, Artificial Sequence
  Description of Artificial Sequence Synthetic peptide
QQEKNEQELAELDKWASLWN

SEQ ID NO: 70 - protein, 20 aa, Artificial Sequence
  Description of Artificial Sequence Synthetic peptide
QQEKNEQELLALDKWASLWN

SEQ ID NO: 71 - protein, 20 aa, Artificial Sequence
  Description of Artificial Sequence Synthetic peptide
QQEKNEQELLEADKWASLWN

SEQ ID NO: 72 - protein, 20 aa, Artificial Sequence
  Description of Artificial Sequence Synthetic peptide
QQEKNEQELLELAKWASLWN

SEQ ID NO: 73 - protein, 20 aa, Artificial Sequence
  Description of Artificial Sequence Synthetic peptide
QQEKNEQELLELDAWASLWN

SEQ ID NO: 74 - protein, 20 aa, Artificial Sequence
  Description of Artificial Sequence Synthetic peptide
QQEKNEQELLELDKAASLWN

SEQ ID NO: 75 - protein, 20 aa, Artificial Sequence
  Description of Artificial Sequence Synthetic peptide
QQEKNEQELLELDKWNSLWN

SEQ ID NO: 76 - protein, 20 aa, Artificial Sequence
  Description of Artificial Sequence Synthetic peptide
QQEKNEQELLELDKWAALWN

SEQ ID NO: 77 - protein, 20 aa, Artificial Sequence
  Description of Artificial Sequence Synthetic peptide
QQEKNEQELLELDKWASLAN

SEQ ID NO: 78 - protein, 20 aa, Artificial Sequence
  Description of Artificial Sequence Synthetic peptide
QQEKNEQELLELDKWASLWA

SEQ ID NO: 79 - protein, 36 aa, Artificial Sequence
  Description of Artificial Sequence Synthetic polypeptide
YTSLIHSLIEESQNQQEKNEQELLELDKWASLWNWF

SEQ ID NO: 80 - DNA, 20 bp, Artificial Sequence
  Description of Artificial Sequence Synthetic oligonucleotide
tccatgacgttcctgacgtt

SEQ ID NO: 81 - DNA, 20 bp, Artificial Sequence
  Description of Artificial Sequence Synthetic primer
agatatcctgcaaggcttct

SEQ ID NO: 82 - DNA, 26 bp, Artificial Sequence
  Description of Artificial Sequence Synthetic primer
tctagaattcaggtccaactgcagca

SEQ ID NO: 83 - DNA, 21 bp, Artificial Sequence
  Description of Artificial Sequence Synthetic primer
gatgtgcagcttcaggagtca

SEQ ID NO: 84 - DNA, 21 bp, Artificial Sequence
  Description of Artificial Sequence Synthetic primer
gaggttcagctgcagcagtct

SEQ ID NO: 85 - DNA, 22 bp, Artificial Sequence
  Description of Artificial Sequence Synthetic primer
cccgtttcagaatggaatgtgc

SEQ ID NO: 86 - DNA, 25 bp, Artificial Sequence
  Description of Artificial Sequence Synthetic primer
gctcaaactttcagaatctaccgac

SEQ ID NO: 87 - DNA, 25 bp, Artificial Sequence
  Description of Artificial Sequence Synthetic primer
agtcagttcgtggaggccggaaatc

SEQ ID NO: 88 - protein, 33 aa, Human immunodeficiency virus
TRPNNNTRKSIHIGPGRAFYTTGEIIGDIRQAH

SEQ ID NO: 89 - protein, 23 aa, Human immunodeficiency virus
TRPNNNTRKSIHIGPGRAFYATE

SEQ ID NO: 90 - protein, 35 aa, Human immunodeficiency virus
KQLQARVLAVERYLKDQQLLGIWGCSGKLICTTAV